(19) United States
(12) Patent Application Publication
(10) Pub. No.: US 2026/0286310 A1
(43) Pub. Date: Sep. 24, 2026

(54) COMPOSITIONS AND METHODS FOR CELL CULTURE APPLICATIONS

(71) Applicant: Rowan University, Glassboro, NJ (US)

(72) Inventors: Andrea Vernengo, Glassboro, NJ (US); Jennifer Weiser, New York, NY (US)

(21) Appl. No.: 19/490,216

(22) PCT Filed: Jun. 4, 2024
(86) PCT No.: PCT/US2024/032431

§ 371 Date: Dec. 4, 2025

Related U.S. Application Data
(60) Provisional application No. 63/471,210, filed on Jun. 5, 2023.

(51) Int. Cl. C12N 5/0775

(52) U.S. Cl. CPC C12N 5/0663; C12N 2533/30

(57) ABSTRACT

The present disclosure generally relates methods and compositions comprising thermosensitive hydrogel compositions that can be used for embedded patterning of cells, cell aggregates, or organoids. The present disclosure further generally relates to methods of inducing cell condensation into geometrically directed tissue structures and simultaneously producing therapeutic cell secretomes.

### CROSS REFERENCE TO RELATED APPLICATIONS

This application claims priority under U.S.C. 35 § 119(e) to U.S. Provisional Application 63/471,210, filed Jun. 5, 2023, the entire contents of which are hereby incorporated by reference.

### BACKGROUND

In general, the field of tissue engineering aims to achieve tissue regeneration through various means, such as in some instances by distributing single cells homogenously across hydrogel scaffolds. Hydrogels, which typically comprise aqueous-based three-dimensional polymer networks, are promising materials for cell encapsulation due in part to their soft, porous, and biocompatible nature. However, traditional approaches where single cells are distributed across milliliter-scale hydrogel volumes are generally not representative of cell distributions of early tissue development, where cells are aggregated into patterned and anisotropic regions of high density prior to sorting, differentiating, and self-assembling into complex tissue structures.

Towards more closely mimicking the contact among cells during the developmental stages, researchers have explored seeding spheroids or organoids within hydrogels. In these approaches, the cell aggregates are suspended in a macromolecular precursor solution, which is then crosslinked around the cell clusters by enzymatic or light-induced crosslinking. The close association of cells within the encapsulated clusters facilitates intercellular communication, promoting differentiation and spontaneous organization of micron-scale tissues. However, the hydrogel formulations often contain static adhesive cues for cells, which causes their spreading and migration out of the cluster assemblies over time in a difficult-to-control fashion. This can inhibit maturation of cell-cell interactions in the early stages of regeneration, which is important for normal tissue development. Moreover, with strategies such as these, the cell aggregates typically are not provided signals to fuse directionally, and therefore have limited ability to fuse into geometrically directed tissue structures.

Freeform printing mediums are an emerging technology for tissue engineering, as they allow for precise spatial placement of concentrated cell suspensions or aggregates into anisotropic patterns within the 3D space of the embedding medium. However, the mediums are generally designed as sacrificial, are not mechanically stable for in vitro culture periods greater than seven days, and generally present static adhesion cues to the cells, thereby conferring limited geometric control over the engineered tissues.

As such, there remains a need in the field for culture platforms capable of freeform spatial placement of concentrated cell or aggregate suspensions.

### BRIEF SUMMARY

In one aspect, the present disclosure generally relates to a thermosensitive hydrogel composition. In certain embodiments, the thermosensitive hydrogel composition comprises a support phase. In certain embodiments, the support phase comprises a primary component.

In certain embodiments, the primary component comprises a continuous or granular polymeric phase dissolved in an aqueous solution. In certain embodiments, the primary component is capable of Bingham plastic rheological behavior in the presence of an aqueous media. In certain embodiments, the support phase comprises a secondary component. In certain embodiments, the secondary component comprises a biocompatible thermally-desolubilizable polymer or co-polymer that exists in an extended form below a critical solution temperature (CST) that is lower than the normal body temperature of a mammal and in a condensed form at or above the CST. In certain embodiments, the support phase comprises an embedded phase. In certain embodiments, the embedded phase comprises at least one uncrosslinked (also known as non-crosslinked) polymer and further comprises at least one cell. In certain embodiments, the embedded phase is capable of extrusion into the support phase at or below the CST of the support phase.

In some aspects, the support phase comprises a homogenous mixture of primary and secondary components dissolved in aqueous solution below the CST of the secondary component. In some aspects, the continuous or granular polymeric phase of the support phase comprises a biocompatible polymeric material. In some aspects, the biocompatible thermally-desolubilizable polymer or co-polymer is in a wetted and/or rod-like state below the CST. In some aspects, the secondary component is capable of releasing or being resistant to cell and protein adhesion below the CST. In some aspects, the biocompatible thermally-desolubilizable polymer or co-polymer is in a globular, hydrophobic, gelated form at or above the CST. In some aspects, the secondary component is capable of being adhesive for cells and proteins at or above the CST. In some aspects, the at least one uncrosslinked polymer of the embedded phase comprises a biocompatible polymer. In some aspects, the at least one uncrosslinked polymer of the embedded phase is dissolved in growth media.

In some aspects, the embedded phase is distributed across the support phase in spatially segregated compartments. In some aspects, the spatially segregated compartments are spherical or cylindrical. In some aspects, the spatially segregated compartments are continuous. In some aspects, the spatially segregated compartments are discontinuous. In some aspects, the spatially defined compartments are anisotropic.

In some aspects, the overall concentration of the continuous or granular polymeric phase dissolved in an aqueous solution of the support phase is from about 0.1% to about 90%. In some aspects, the continuous or granular polymeric phase dissolved in an aqueous solution is selected from the group consisting of poly(ethylene oxides), poly(propylene oxides), copolymers of PEO and polylactic acid (PLA), polyvinyl alcohol, celluloses, agar, agarose, chitosan, alginate, collagen, celluloses, polyacrylic acid, hyaluronates, keratins, and decellularized tissue components from any one of intervertebral disc, tendon, ligaments, cartilage, bone, cardiac tissues, and vascular tissues. In some aspects, the secondary component of the support phase comprises at least one material that is distinct from the primary component of the support phase. In some aspects, the overall concentration of the secondary component is about 1% to about 25%. In some aspects, the secondary component comprises at least one polymer selected from the group consisting of poly(N-isopropyl acrylamides) (PNIPAAm), poly(N,N-diethylacrylamide), poly(N-vinylcaprolactam), poly(2-oxazolines), poly(2-dimethylamino)ethyl methacrylate), and poloxamers ((poly(ethylene oxide) (PEO)-b-poly(propylene oxide)-b-PEO). In some aspects, the biocompatible thermally-desolubilizable polymer or co-polymer of the secondar component is covalently linked to a polymer selected from the group consisting of PEO, polylactic-coglycolic acid, alginate, hyaluronic acid, gelatin, collagen, chondroitin sulfate, and decellularized extracellular matrix components. In some aspects, the decellularized extracellular matrix components are derived from tendon, ligament, bone, cartilage, intervertebral disc, vascular tissues, and/or cardiac tissues. In some aspects, the secondary component comprises PNIPAAm. In some aspects, the PNIPAAm is covalently linked to chondroitin sulfate. In some aspects, the secondary component comprises PNIPAAm at an overall concentration of about 1% to about 25%. In some aspects, the support phase comprises a yield point of 50-1000 Pa below the normal body temperature of the mammal. In some aspects, the support phase exhibits at least 25% greater elastic modulus above the CST of the secondary component. In some aspects, the embedded phase comprises at least one of gelatin, collagen, chitosan, alginate, collagen, cellulose, modified celluloses, and soluble decellularized tissue components. In some aspects, the modified cellulose is carboxymethyl cellulose, methyl cellulose, and/or hydroxypropylmethyl cellulose. In some aspects, the soluble decellularized tissue components are derived from at least one of intervertebral disc, tendon, ligaments, cartilage, bone, cardiac tissues, and/or vascular tissues. In some aspects, the soluble decellularized tissue components are dissolved in growth media. In some aspects, the embedded phase has a viscosity between 10 and 1×10⁷ Pa-S.

In some aspects, the at least one cell comprises a suspending living cell, allogenic mesenchymal stem cell, and/or induced pluripotent stem cell. In some aspects, the induced pluripotent stem cell is capable of inducing at least one cellular process related to nucleus pulposus, annulus fibrosus, hyaline cartilage, elastic cartilage, fibrocartilage, tendon, ligament, long bones, short bones, flat bone, irregular bones, cardiac tissue, or vascular tissue.

In some aspects, the embedded phase comprises cylindrical channels. In some aspects, the channels are from about 10 μm to about 1000 μm in diameter. In some aspects, the embedded phase comprises spherical compartments. In some aspects, the spherical compartments are connected within the support phase. In some aspects, the spherical compartments are not connected within the support phase.

Moreover, in one aspect, the present disclosure generally relates to a method for culturing a cell. In certain embodiments, the method comprises providing a thermosensitive hydrogel composition. In certain embodiments, the thermosensitive hydrogel composition comprises a support phase. In certain embodiments, the support phase comprises a primary component. In certain embodiments, the primary component comprises a continuous or granular polymeric phase dissolved in an aqueous solution. In certain embodiments, the primary component is capable of Bingham plastic rheological behavior in the presence of an aqueous media. In certain embodiments, the support phase comprises a secondary component. In certain embodiments, the secondary component comprises a biocompatible thermally-desolubilizable polymer or co-polymer that exists in an extended form below a critical solution temperature (CST) that is lower than the normal body temperature of the mammal and in a condensed form at or above the CST. In certain embodiments, the method comprises depositing an embedded phase into the support phase at or below the CST of the biocompatible thermally-desolubilizable polymer or co-polymer of the support phase. In certain embodiments, the embedded phase comprises at least one uncrosslinked polymer and further comprises a cell. In certain embodiments, the method comprises culturing the at least one cell, wherein during the culturing, the temperature of the composition is increased to at or above the CST of the biocompatible thermally-desolubilizable polymer or co-polymer of the support phase following deposition of the embedded phase. In certain embodiments, the method comprises optionally collecting the secretome expressed by the cell.

In some aspects, the cell comprises a single cell. In some aspects, the cell comprises more than one cell. In some aspects, the temperature of the hydrogel composition comprising the embedded phase is decreased to below the CST during the culturing phase. In some aspects, the temperature of the hydrogel composition comprising the embedded phase is cycled between the temperature being greater than or equal to the CST and the temperature being less than the CST at least once during the culturing phase. In some aspects, the temperature is cycled between at least 1 time and at least 1,000 times during the culturing phase. In some aspects, the temperature is cycled not more than 3 times per week of the culture phase. In some aspects, the time period for each cycle is between about 15 minutes to about 5 hours. In some aspects, a secretome expressed by the cell is collected. In some aspects, lowering the temperature below the CST promotes reversible de-adhesion of proteins and cells from the support phase. In some aspects, the temperature is cycled between about 4° C. and about 37° C. In some aspects, the temperature is cycled between about 25° C. and about 37° C.

In some aspects, the support phase comprises a homogenous mixture of primary and secondary components dissolved in aqueous solution below the CST of the secondary component. In some aspects, the continuous or granular polymeric phase of the support phase comprises a biocompatible polymeric material. In some aspects, the biocompatible thermally-desolubilizable polymer or co-polymer is in a wetted and/or rod-like state below the CST. In some aspects, the secondary component is capable of releasing or being resistant to cell and protein adhesion below the CST. In some aspects, the biocompatible thermally-desolubilizable polymer or co-polymer is in a globular, hydrophobic, gelated form at or above the CST. In some aspects, the secondary component is capable of being adhesive for cells and proteins at or above the CST. In some aspects, the at least one uncrosslinked polymer of the embedded phase comprises a biocompatible polymer. In some aspects, the at least one uncrosslinked polymer of the embedded phase is dissolved in growth media.

In some aspects, the embedded phase is distributed across the support phase in spatially segregated compartments. In some aspects, the spatially segregated compartments are spherical or cylindrical. In some aspects, the spatially segregated compartments are continuous. In some aspects, the spatially segregated compartments are discontinuous. In some aspects, the spatially defined compartments are anisotropic.

In some aspects, the overall concentration of the continuous or granular polymeric phase dissolved in an aqueous solution of the support phase is from about 0.1% to about 90%. In some aspects, the continuous or granular polymeric phase dissolved in an aqueous solution is selected from the group consisting of poly(ethylene oxides), poly(propylene oxides), copolymers of PEO and polylactic acid (PLA), polyvinyl alcohol, celluloses, agar, agarose, chitosan, alginate, collagen, celluloses, polyacrylic acid, hyaluronates, keratins, and decellularized tissue components from any one of intervertebral disc, tendon, ligaments, cartilage, bone, cardiac tissues, and vascular tissues. In some aspects, the secondary component of the support phase comprises at least one material that is distinct from the primary component of the support phase. In some aspects, the overall concentration of the secondary component is about 1% to about 25%. In some aspects, the secondary component comprises at least one polymer selected from the group consisting of poly(N-isopropyl acrylamides) (PNIPAAm), poly(N,N-diethylacrylamide), poly(N-vinylcaprolactam), poly(2-oxazolines), poly(2-dimethylamino)ethyl methacrylate), and poloxamers ((poly(ethylene oxide) (PEO)-b-poly(propylene oxide)-b-PEO). In some aspects, the biocompatible thermally-desolubilizable polymer or co-polymer of the secondar component is covalently linked to a polymer selected from the group consisting of PEO, polylactic-coglycolic acid, alginate, hyaluronic acid, gelatin, collagen, chondroitin sulfate, and decellularized extracellular matrix components. In some aspects, the decellularized extracellular matrix components are derived from tendon, ligament, bone, cartilage, intervertebral disc, vascular tissues, and/or cardiac tissues. In some aspects, the secondary component comprises PNIPAAm. In some aspects, the PNIPAAm is covalently linked to chondroitin sulfate. In some aspects, the secondary component comprises PNIPAAm at an overall concentration of about 1% to about 25%. In some aspects, the support phase comprises a yield point of 50-1000 Pa below the normal body temperature of the mammal. In some aspects, the support phase exhibits at least 25% greater elastic modulus above the CST of the secondary component. In some aspects, the embedded phase comprises at least one of gelatin, collagen, chitosan, alginate, collagen, cellulose, modified celluloses, and soluble decellularized tissue components. In some aspects, the modified cellulose is carboxymethyl cellulose, methyl cellulose, and/or hydroxypropylmethyl cellulose. In some aspects, the soluble decellularized tissue components are derived from at least one of intervertebral disc, tendon, ligaments, cartilage, bone, cardiac tissues, and/or vascular tissues. In some aspects, the soluble decellularized tissue components are dissolved in growth media. In some aspects, the embedded phase has a viscosity between 10 and 1×10⁷ Pa-S.

In some aspects, the at least one cell comprises a suspending living cell, allogenic mesenchymal stem cell, and/or induced pluripotent stem cell. In some aspects, the induced pluripotent stem cell is capable of inducing at least one cellular process related to nucleus pulposus, annulus fibrosus, hyaline cartilage, elastic cartilage, fibrocartilage, tendon, ligament, long bones, short bones, flat bone, irregular bones, cardiac tissue, or vascular tissue.

In some aspects, the embedded phase comprises cylindrical channels. In some aspects, the channels are are from about 10 μm to about 1000 μm in diameter. In some aspects, the embedded phase comprises spherical compartments. In some aspects, the spherical compartments are connected within the support phase. In some aspects, the spherical compartments are not connected within the support phase. In some aspects, the method further comprises d. collecting the secretome expressed by the at least one cell.

Moreover, in one aspect, the present disclosure generally relates to a method of collecting a secretome of a cell. In certain embodiments, the method comprises providing a thermosensitive hydrogel composition. In certain embodiments, the thermosensitive hydrogel composition comprises a support phase. In certain embodiments, the support phase comprises a primary component. In certain embodiments, the primary component comprises a continuous or granular polymeric phase dissolved in an aqueous solution. In certain embodiments, the primary component is capable of Bingham plastic rheological behavior in the presence of an aqueous media. In certain embodiments, the support phase comprises a secondary component. In certain embodiments, the secondary component comprises a biocompatible thermally-desolubilizable polymer or co-polymer that exists in an extended form below a critical solution temperature (CST) that is lower than the normal body temperature of the mammal and in a condensed form at or above the CST. In certain embodiments, the method comprises depositing an embedded phase into the support phase at or below the CST of the biocompatible thermally-desolubilizable polymer or co-polymer of the support phase. In certain embodiments, the embedded phase comprises at least one uncrosslinked polymer and further comprises a cell. In certain embodiments, the method comprises culturing the at least one cell. In certain embodiments, during the culturing, the temperature of the composition is increased to at or above the CST of the biocompatible thermally-desolubilizable polymer or co-polymer of the support phase following deposition of the embedded phase. In certain embodiments, the method comprises collecting the secretome expressed by the at least one cell.

### BRIEF DESCRIPTION OF THE SEVERAL VIEWS OF THE DRAWING

FIG. 1 is a flowchart summarizing an embodiment of a hydrogel composition and an embodiment of a method of application of the hydrogel composition in accordance with Example 1.

FIG. 2A-FIG. 2D present the results related to the rheological properties of an exemplary hydrogel composition comprised of aqueous solutions of 3% (w/v) Poly(N-isopropylacrylamide)-chondroitin sulfate-A (pNIPAAm-CS)+0.8% (w/v) poly acrylic acid (PAA)+1% gelatin (experimental group, grey) compared to 0.8% (w/v) PAA (conventional freeform printing medium, control group, black) in accordance with Example 2. Average values are plotted. FIG. 2A presents a graphical representation of an amplitude sweep test from 0.01% to 1000% oscillatory strain. FIG. 2B presents a graphical representation of a step strain test with oscillatory strain alternating between 1% strain (low) and 250% strain (high). FIG. 2C presents a graphical representation of shear-thinning behavior for the experimental and control groups with a rotational viscosity test applying a shear rate from 0.01 s⁻¹ to 100 s⁻¹. FIG. 2D presents a graphical representation of a temperature ramp test from 25° C. to 37° C. with 1% constant strain. Vertical dashed lines indicate the change in temperature.

FIG. 3A-FIG. 3D present schematics and results related to the characterization of freeform 3D print fidelity inside 3% (w/v) pNIPAAm-CS+0.8% (w/v) PAA+1% (w/v) gelatin (experimental group, grey) and 0.8% (w/v) PAA (freeform printing control, black) in accordance with Example 3. FIG. 3A presents a schematic representation of the digitally designed grid with the dimensions designated for quantification of print fidelity: (i) 117° angle, and (ii) Pr value. FIG. 3B presents digital images of a grid comprised of 6% (w/v) gelatin embedded within an exemplary hydrogel composition and control group. FIG. 3C presents a graphical representation of angle quantification of the freeform printed grids within the experimental and control groups. FIG. 3D presents a graphical representation of Pr value of the freeform printed grids within the experimental and control groups.

FIG. 4A-FIG. 4D present schematics and results related to small angle x-ray scattering analysis of exemplary thermally sensitive hydrogel compositions in accordance with Example 4. FIG. 4A presents a graphical representation of Guinier analysis of a dilute thermoresponsive hydrogel composition (0.3% pNIPAAm, 0.08% CP and 0.01% gelatin) at two different temperatures (25° C., circles; 37° C., squares). FIG. 4B presents a schematic representation of a rod-like, wetted state of an exemplary thermally sensitive hydrogel composition. FIG. 4C presents a schematic representation of a thermally sensitive hydrogel composition in a collapsed entangled globule with a rough surface. FIG. 4D presents a graphical representation of the non-thermoresponsive hydrogel without the pNIPAAM.

FIG. 5A-FIG. 5D present schematics and results related to cell culture studies conducted with human bone marrow-derived mesenchymal stromal cells (BM-MSCs) embedded within 3% (w/v) pNIPAAm-CS+0.8% (w/v) PAA+1% (w/v) gelatin hydrogels for five weeks under adhesive or on-off adhesive conditions in accordance with Example 5. FIG. 5A presents a schematic representation of BM-MSC embedded within 6% gelatin and deposited by extrusion 3D printing as three circles with a radius of 5 mm, stacked in Z-direction with 1 mm spacing, and deposited starting height of 2 mm inside the hydrogel. FIG. 5B presents graphical representation of the on-off adhesive conditions, where the cell culture was cycled below the critical solution temperature (CST) to 25° C. for 15 minutes every 5 days, and the adhesive conditions, where the temperature was maintained above the CST at 37° C. for 35 days. FIG. 5C presents images of cell nuclei stained with DAPI and TRITC-conjugated phalloidin of the cells at day 35 of culture, with white arrows indicating spread cells and grey arrows indicating migrating cells. The scale bars of FIG. 5C are 100 μm scale bars. FIG. 5D presents a graphical representation of the semi-quantitative analysis of the images demonstrating the width of the area of cell placement.

FIG. 6A-FIG. 6B present macroscopic and confocal images related to geometrically directed and anisotropic cell and tissue structures assembled by 5 weeks of on-off adhesive culture within an exemplary thermosensitive hydrogel composition in accordance with Example 5. FIG. 6A presents images of human bone marrow derived mesenchymal stromal cells (BM-MSC) that produced stable ring or necklace like tissue construct. FIG. 6B presents an image of L929 murine fibroblasts that produced a grid-like tissue construct.

FIG. 7A-FIG. 7E present fluorescent confocal images of human bone marrow-derived mesenchymal stromal cells (BM-MSCs) aggregates (250,000 cells per aggregate) embedded within 3% (w/v) pNIPAAm-CS+0.8% (w/v) PAA+1% (w/v) gelatin hydrogels for five weeks under adhesive or on-off adhesive conditions in accordance with Example 6. FIG. 7A presents a fluorescent confocal image taken at the Day 0 timepoint. FIG. 7B present a fluorescent confocal image taken at the Day 7 timepoint for on-off adhesive conditions. FIG. 7C presents a fluorescent confocal image taken at the Day 7 time point for adhesive conditions. FIG. 7D presents a fluorescent confocal image taken at the Day 35 timepoint for on-off adhesive conditions. FIG. 7E presents a fluorescent confocal image taken at the Day 35 timepoint for adhesive conditions.

FIG. 8A-FIG. 8F depict the results of the rheological characterization of aqueous solutions of 1%, 3%, and 5% (w/v) pNIPAAm-CS+0.8% (w/v) PAA compared to 0.8% (w/v) PAA (control) in accordance with Example 7. Average values are plotted. FIG. 8A presents a graphical representation of an amplitude sweep test from 0.01% to 1000% oscillatory strain. FIG. 8B presents a graphical representation of flow stress values, taken to be the amplitude sweep curve cross-over of G′ and G″. FIG. 8C presents a graphical representation of a step strain test with oscillatory strain alternating between 1% strain (low) and 250% strain (high). FIG. 8D presents a graphical representation of a temperature ramp test from 25° C. to 37° C. with 1% constant strain. Vertical dashed lines indicate the change in temperature. FIG. 8E presents a graphical representation of a temperature-triggered percentage increase in G. FIG. 8F presents a graphical representation of shear-thinning behaviour determined with rotational viscosity test with a shear rate from 0.01 s-1 to 100 s-1.

FIG. 9 presents a heat map representing a cytokine profile of bone marrow-derived MSCs in accordance with Example 9.

### DETAILED DESCRIPTION

In one aspect, the present disclosure relates to thermosensitive hydrogel compositions for embedded patterning of cells, cell aggregates, and/or organoids. In another aspect, the present disclosure relates to methods of inducing the condensation of cells into geometrically directed tissue structures and the simultaneous production of therapeutic cell secretomes.

As discussed above, there remains a need in the field for culture platforms capable of freeform spatial placement of concentrated cells, their long-term culture, control over adhesion and de-adhesion cues over the culture period, and the capability of harvesting secretomes. Though freeform printing mediums are an emerging technology for tissue engineering, the mediums are generally designed as sacrificial, are not mechanically stable for in vitro culture.

As such, the present disclosure generally relates to a thermosensitive hydrogel composition for culturing a cell. Furthermore, the present disclosure also generally encompasses methods of using a thermosensitive hydrogel composition for culturing a cell, and subsequently optionally collecting a secretome from the cell.

In some aspects, the thermosensitive hydrogel compositions described herein represent 3D culture platforms which are capable of freeform spatial placement of one or more cells, their long-term culture, e.g., at least 5 weeks, and control over adhesion and de-adhesion cues over the culture period. Moreover, in some aspects, the thermosensitive hydrogel compositions described herein represent a non-sacrificial freeform printing medium that can mechanically stabilize an embedded compartment of high cell density. In some aspects, the thermosensitive hydrogel compositions described herein are capable of supporting spatial segregation of embedded cells, cell aggregates, and/or organoids. For instance, in some aspects, organoids can be gradually shaped over time from a round to oblong morphology and stimulated to fuse. In some instances, such organoids can express vastly different genomic and proteomic profiles as compared to those cultured under adhesive conditions, such as those adhesive conditions described herein, or as compared with other methods used in the field.

As further described infra, in some aspects the present disclosure relates to methods of culturing a cell comprising use of the thermosensitive hydrogel compositions and further collecting the secretome of the cell. In some aspects of such methods, the use of the thermosensitive hydrogel compositions for culturing allows for a user to guide certain aspects of a tissue regeneration process, such as cell-cell binding maturity, cell-matrix binding and spreading, and directionally oriented cell aggregate fusion. In some aspects, the methods described herein can promote condensation of the cell or cells cultured in the thermosensitive hydrogel composition. Such condensation can be used for the purposes of, for example, assembling geometric directed, fused tissue structures while inducing production of therapeutic secretomes by the cells. In some aspects, the secretome of the cultured cell or cells is collected, and the collected secretome from such cultured cell(s) can be harnessed for disease-modifying and therapeutic properties. The collection of the secretome from such cultured cells represents a sizable advance over traditional scaffold compositions in terms of potential to engineer complex, anisotropic, and scalable tissue structures and therapeutic secretomes in vitro.

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, protein and nucleic acid chemistry, and nucleic acid hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-know-n and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “therapeutic” as used herein means a treatment and/or prophylaxis. A therapeutic effect is obtained by any degree of suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.3, and 6. This applies regardless of the breadth of the range.

As used herein, the term “polymer” refers to the polymerization product of two or more monomers and is inclusive of homo-, co-, ter-, tetra-polymers, and so forth Unless indicated to the contrary herein, the term polymer includes oligomers.

As used herein, the term “thermally sensitive hydrogel,” “thermosensitive hydrogel,” “thermally responsive hydrogel,” and “thermoresponsive hydrogel” are used interchangeably and generally refer to a hydrogel comprising at least one thermally-desolubilizable polymer.

As used herein, the term “thermally-desolubilizable polymer” generally refers to a polymer that will undergo a phase transition from an extended, more soluble (i.e., more suspendable) form to a compacted, less soluble (i.e., less suspendable; or even essentially completely insoluble) form when the temperature of an aqueous suspension of the polymer is raised above a critical solution temperature that is a characteristic of the polymer.

As used herein, a material or composition that is “capable of Bingham plastic rheological behavior” generally refers to a material or composition that comprises a viscoelastic material that behaves as a rigid body below a threshold stress value (i.e., yield stress) but flows as a viscous fluid at above the threshold value. The viscosity of the fluid above the threshold stress value decreases under increasing strain, facilitating translation of an extrusion device such as a needle, cannula, or aspiration tip through the gel (i.e. shear thinning). As such, materials or compositions that are capable of Bingham plastic rheological behavior are generally considered to be materials or compositions capable of extrusion.

Additionally, such Bingham plastic materials can also exhibit reformation of solid-like behavior immediately after local removal of the translating extrusion device (i.e. self-healing). In some instances, the mechanical properties of the reformed solid can be strong enough to prevent deformation of a soft embedded material deposited within its structure via extrusion device. In some aspects, a primary component of a support phase of the present disclosure is capable of Bingham plastic rheological behavior.

As used herein, the term “biocompatible” generally refers to a material that does not induce a medically-significant adverse pathological event upon implantation of the material at a location in or on the body of a subject, such as a mammal.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is human.

### Thermosensitive Hydrogel Compositions

In one aspect, the present disclosure generally relates to a thermosensitive hydrogel composition, wherein the thermosensitive hydrogel composition comprises: a) a support phase, wherein the support phase comprises: i) a primary component; and ii) a secondary component; and b) an embedded phase. In a second aspect, the present disclosure generally relates to a thermosensitive hydrogel composition, wherein the thermosensitive hydrogel composition comprises: a) a support phase, wherein the support phase comprises: i) a primary component, wherein the primary component comprises a continuous or granular polymeric phase dissolved in an aqueous solution, further wherein the primary component is capable of Bingham plastic rheological behavior in the presence of an aqueous media; ii) a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b) an embedded phase, wherein the embedded phase comprises at least one uncrosslinked polymer and further comprises at least one cell, wherein the embedded phase is capable of extrusion into the support phase at or below the CST of the support phase.

### Support Phase

In some aspects, the thermosensitive hydrogel composition comprises a support phase, wherein the support phase comprises a primary component and a secondary component. In some aspects, the support phase comprises a homogenous mixture of primary and secondary components dissolved in aqueous solution below the CST of the secondary component. In some aspects, the support phase exhibits a yield point of 50-1000 Pa below the normal body temperature of the mammal. In some aspects, the support phase exhibits at least 25% greater elastic modulus above the CST of the secondary component.

In some aspects, the primary component comprises a continuous or granular polymeric phase dissolved in an aqueous solution. In some aspects, the primary component is capable of Bingham plastic rheological behavior in the presence of an aqueous media. In some aspects, the continuous or granular polymeric phase of the support phase comprises a biocompatible polymeric material. In some aspects, the continuous or granular polymeric phase is selected from the group consisting of poly(ethylene oxides), poly(propylene oxides), copolymers of PEO and polylactic acid (PLA), polyvinyl alcohol, celluloses, agar, agarose, chitosan, alginate, collagen, celluloses, polyacrylic acid, hyaluronates, keratins, and decellularized tissue components from intervertebral disc, tendon, ligaments, cartilage, bone, cardiac tissues, and vascular tissues. In some aspects, the overall concentration of the continuous or granular polymeric phase dissolved in an aqueous solution of the support phase is from about 0.1% to about 90%. In some aspects, non-covalent interactions between dissolved chains can provide yield stress, shear thinning, and/or self-healing properties to the composition.

In some aspects, the primary component of the support phase exhibiting Bingham plastic behavior is a bulk polymeric phase dissolved in a aqueous solvent. In some aspects, the bulk polymer can be synthesized by polymerization of monomers, biosynthesized by bacterial cultures, and/or isolated as from naturally-derived source such as plants or animals. In some aspects, the Bingham plastic properties can arise by use of a granular medium comprised of nano or micron-sized crosslinked polymeric networks swelled in an aqueous solvent in high enough overall concentrations (0.1 to 90%) such that non-covalent physical interactions between granules provide the yield stress, shear thinning, and self-healing properties. Granular mediums are generated by means of fragmenting aqueous solvent based hydrogel networks by mechanical disruption.

In some aspects, the secondary component comprises a biocompatible thermally-desolubilizable polymer or co-polymer. In some aspects, the biocompatible thermally-desolubilizable polymer or co-polymer exists in an extended form below a critical solution temperature (CST) that is lower than the normal body temperature of the mammal and in a condensed form at or above the CST. In some aspects, the biocompatible thermally-desolubilizable polymer or co-polymer is in a wetted and/or rod-link state below the CST. In some aspects, the secondary component is capable of releasing or being resistant to cell and protein adhesion below the CST. In some aspects, the biocompatible thermally-desolubilizable polymer or co-polymer is in a globular, hydrophobic, gelated form at or above the CST. In some aspects, the secondary component is capable of being adhesive for cells and proteins at or above the CST.

In some aspects, the secondary component of the support phase comprises at least one material that is distinct from the primary component of the support phase. In some aspects, the overall concentration of the secondary component is about 1% to about 25%. In some aspects, the secondary component comprises at least one polymer selected from the group consisting of poly(N-isopropyl acrylamides) (PNIPAAm), poly(N,N-diethylacrylamide), poly(Nvinylcaprolactam), poly(2-oxazolines), poly(2-dimethylamino)ethyl methacrylate), and poloxamers ((poly(ethylene oxide) (PEO)-b-poly(propylene oxide)-b-PEO). In some aspects, the secondary component is covalently linked or blended with one or more polymers is selected from the group consisting of PEO, poly(lactic-coglycolic acid, alginate, hyaluronic acid, gelatin, collagen, chondroitin sulfate, and decellularized extracellular matrix components from tendon, ligament, bone, cartilage, intervertebral disc, vascular or cardiac tissues.

In some aspects, the biocompatible thermally-desolubilizable polymer or co-polymer of the secondary component is covalently linked to a polymer selected from the group consisting of PEO, polylactic-coglycolic acid, alginate, hyaluronic acid, gelatin, collagen, chondroitin sulfate, and decellularized extracellular matrix components. For instance, the decellularized extracellular matrix components can be derived from tendon, ligament, bone, cartilage, intervertebral disc, vascular or cardiac tissues. In some aspects, the secondary component comprises PNIPAAm. In some aspects, the secondary component comprises PNIPAAm, and the PNIPAAm is covalently linked to chondroitin sulfate. In some aspects, the pNIPAAm grafted with chondroitin (pNIPAAM-CS) using a molar ratio of NIPAAm monomer to chondroitin sulfate of 2500:1. In some aspects, the secondary component comprises PNIPAAm at an overall concentration of about 1% to about 25%. In some aspects, the secondary component comprises PNIPAAm-CS. In some aspects, the secondary component comprises PNIPAAm-CS at an overall concentration of about 1% to about 25%.

In some aspects, the primary and secondary components of the support phase are present in relative amounts such that the primary component retains a yield stress, shear thinning, and self-healing properties. In some aspects, the primary and secondary components of the support phase mixture are present in relative amounts such that the secondary component retains CST induced gelation below normal body temperature of a mammal. In some aspects, the support phase exhibits a yield point of 50-2000 Pa below the CST. In some aspects, the support phase exhibits at least a 25% increase in elastic modulus upon heating at or above the CST to induce gelation. In some aspects, the support phase comprises pNIPAAm-CS, PAA, and/or gelatin. In some aspects, the support phase comprises pNIPAAm-CS at an overall concentration of about 1% to about 25%, PAA at an overall concentration of about 0.1% to about 90%, and/or gelatin at a concentration of about 0.1% to about 90%. In some aspects, the support phase comprises 3% (w/v) pNIPAAm-CS+0.8% (w/v) PAA+1% (w/v) gelatin.

### Embedded Phase

In some aspects, the embedded phase of the thermosensitive hydrogel composition comprises at least one uncrosslinked polymer. In some aspects, the embedded phase comprises at least one cell. In some aspects, the embedded phase is capable of extrusion into the support phase at or below the CST of the support phase. In some aspects, the at least one uncrosslinked polymer of the embedded phase comprises a biocompatible polymer. In some aspects, the at least one uncrosslinked polymer of the embedded phase is dissolved in growth media. In some aspects, the embedded phase comprises a cell-laden material placed as spatially segregated compartments distributed in anisotropic patterns across the support phase. In some aspects, the embedded phase comprises a cell-laden material placed across the support phase on top of the support phase. In some aspects, the embedded phase comprises a cell laden material placed across the support phase near the top of the support phase. In some aspects, the embedded phase is dispensed by manual or automatic extrusion into the support phase at or below the CST. In some aspects, the embedded phase comprises suspended single cells, cell aggregates, or organoids. In some instances, the embedded material exhibits sufficient high viscosity to prevent flow away from the location in which it was extruded, but sufficient low viscosity to flow through a cannula, needle or aspiration tip for extrusion. In some aspects, the embedded phase is crosslinked after deposition. In some aspects, the embedded phase is not crosslinked after deposition.

In some aspects, the embedded phase has a viscosity between 10 and 1×10⁷ Pa-S. In some aspects, the embedded phase is disturbed across the support phase in spatially segregated compartments. In some aspects, the spatially segregated compartments are spherical or are cylindrical. In some aspects, wherein the spatially segregated compartments are continuous or discontinuous. In some aspects, the spatially segregated compartments are dispensed on top of the support phase. In some aspects, the spatially segregated compartments are dispensed on top of the support phase in anisotropic patterns. In some aspects, the spatially defined compartments are anisotropic. In some aspects, the embedded phase comprises cylindrical channels. In some aspects, the channels are at least 100 μm in diameter. In some aspects, the embedded phase comprises spherical compartments. In some aspects, the spherical compartments are connected within the support phase. In some aspects, the spherical compartments are not connected within the support phase. In some aspects, the spherical compartments are not connected on top of the support phase. In some aspects, the compartments of the embedded phase comprise spatially defined regions of high cell or tissue concentration within the hydrogel composition. In some aspects, the compartments of the embedded phase comprise spatially defined regions of high cell or tissue concentration on top of the hydrogel composition. In some aspects, the compartments of the embedded phase comprise spatially defined regions of high cell or tissue concentration near the top of the hydrogel composition. In some aspects, the minimum diameter for the embedded compartment is related to the extrusion or aspiration tip. In some aspects, minimum diameter for the embedded compartment is related to the tip radius, the flowrate through the tip, and viscosity of the soft embedded phase. In some aspects, the embedded phase can be extruded within the composite gel phase with a stationary tip to form an embedded compartment within the support phase. In some aspects, the embedded phase can be extruded on top of the composition gel phase so as to form a second layer of embedded phase on top of the support phase. In some aspects, the compartments are cylindrical channels placed in a desired anisotropic pattern. In some aspects, the compartments are cylindrical channels sitting on top of the support phase. In some aspects, embedded compartments within the support phase can be spaced at any desired distance from one another. In some aspects, embedded compartments may be discrete or connected with one another. In some aspects, the diameter of the embedded compartment is from about 5 microns to about 2500 microns, and any and all values therebetween. In some aspects, the diameter of the embedded compartment is from about 100 micron to about 1000 micron. In some aspects, the diameter of the embedded compartments are at least 100 microns.

In some aspects, the embedded phase is distributed on top of the support phase. In some aspects, the embedded phase is placed near the top of the support phase, such that the channels are not completely embedded in the support phase.

In some aspects, the embedded phase comprises at least one of gelatin, collagen, chitosan, alginate, collagen, cellulose, modified celluloses, or soluble decellularized tissue components. For instance, the modified cellulose can be carboxymethyl cellulose, methyl cellulose, or hydroxypropylmethyl cellulose. In some aspects, the soluble decellularized tissue components are derived from intervertebral disc, tendon, ligaments, cartilage, bone, cardiac or vascular tissues. In some instances, the soluble decellularized tissue components are dissolved in growth media. In some aspects, the embedded phase comprises a material that may be cured to increase long term stability during cell culture.

In some aspects, the at least one cell comprises suspending living cells, allogenic mesenchymal stem cells, mesenchymal stromal cells and/or induced pluripotent stem cells. In some aspects, the at least one cell is derived from a plant, fungi, or algae. In some aspects, the at least one cell is derived from medicinal mushrooms, medicinal mycological cultures, or therapeutic fungi, such shiitake mushrooms. In some aspects, the at least one cell is derived from an aquatic plant, such as, for instance, Spirulina algae and aloe vera. In some aspects, the at least one cell is derived from a plant known for medicinal qualities such as, for instance, Ocimum basilicum, Curcuma longa, Zingiber officinale, and Vitis vinifera. In some aspects, the induced pluripotent stem cells are capable of inducing at least one cellular process related to nucleus pulposus, annulus fibrosus, hyaline cartilage, elastic cartilage, fibrocartilage, tendon, ligament, long bones, short bones, flat bone, irregular bones, cardiac tissue, or vascular tissue. In some aspects, the cell comprises a single cell. In some aspects, the cell comprises more than one cell. In some aspects, the cell comprise single cells, cell aggregates, or organoids. In some aspects, the cell type comprises nucleus pulposus, annulus fibrosus, hyaline cartilage, elastic cartilage, fibrocartilage, tendon, ligament, long bones, short bones, flat bone, irregular bones, cardiac tissue, or vascular tissue.

### Cell Culture Methods

In another aspect, the present disclosure generally relates to a method for culturing a cell, wherein the method comprises a) providing a thermosensitive hydrogel composition comprising a support phase and an embedded phase, such as a thermosensitive hydrogel composition comprising a support phase and an embedded phase as described herein; b) depositing an embedded phase into the support phase at or below a critical solution temperature (CST) of the support phase, wherein the embedded phase comprises at least one uncrosslinked polymer and further comprises a cell; and c) culturing the at least one cell, wherein during the culturing, the temperature of the composition is increased to at or above the CST of the support phase following deposition of the embedded phase. In one aspect, the present disclosure relates to a method for culturing a cell, wherein the method comprises a) providing a thermosensitive hydrogel composition comprising a support phase and an embedded phase, such as a thermosensitive hydrogel composition comprising a support phase and an embedded phase as described herein: b) depositing an embedded phase near the top of the support phase at or below a critical solution temperature (CST) of the support phase, wherein the embedded phase comprises at least one uncrosslinked polymer and further comprises a cell; and c) culturing the at least one cell, wherein during the culturing, the temperature of the composition is increased to at or above the CST of the support phase following deposition of the embedded phase. In one aspect, the present disclosure relates to a method for culturing a cell, wherein the method comprises a) providing a thermosensitive hydrogel composition comprising a support phase and an embedded phase, such as a thermosensitive hydrogel composition comprising a support phase and an embedded phase as described herein: b) depositing an embedded phase on the top of the support phase at or below a critical solution temperature (CST) of the support phase, wherein the embedded phase comprises at least one uncrosslinked polymer and further comprises a cell; and c) culturing the at least one cell, wherein during the culturing, the temperature of the composition is increased to at or above the CST of the support phase following deposition of the embedded phase.

In another aspect, the present disclosure generally relates to a method for culturing a cell, wherein the method comprises: a) providing a thermosensitive hydrogel composition, wherein the thermosensitive hydrogel composition comprises: i) a support phase, wherein the support phase comprises: 1) a primary component, wherein the primary component comprises a continuous or granular polymeric phase dissolved in an aqueous solution, further wherein the primary component is capable of Bingham plastic rheological behavior in the presence of an aqueous media; and 2) a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b) depositing an embedded phase into the support phase at or below the CST of the biocompatible thermally-desolubilizable polymer or co-polymer of the support phase, wherein the embedded phase comprises at least one uncrosslinked polymer and further comprises a cell; and c) culturing the at least one cell, wherein during the culturing, the temperature of the composition is increased to at or above the CST of the biocompatible thermally-desolubilizable polymer or co-polymer of the support phase following deposition of the embedded phase. In another aspect, the present disclosure generally relates to a method for culturing a cell, wherein the method comprises: a) providing a thermosensitive hydrogel composition, wherein the thermosensitive hydrogel composition comprises: i) a support phase, wherein the support phase comprises: 1) a primary component, wherein the primary component comprises a continuous or granular polymeric phase dissolved in an aqueous solution, further wherein the primary component is capable of Bingham plastic rheological behavior in the presence of an aqueous media; and 2) a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b) depositing an embedded phase near the top of the support phase at or below the CST of the biocompatible thermally-desolubilizable polymer or co-polymer of the support phase, wherein the embedded phase comprises at least one uncrosslinked polymer and further comprises a cell; and c) culturing the at least one cell, wherein during the culturing, the temperature of the composition is increased to at or above the CST of the biocompatible thermally-desolubilizable polymer or co-polymer of the support phase following deposition of the embedded phase. In another aspect, the present disclosure generally relates to a method for culturing a cell, wherein the method comprises: a) providing a thermosensitive hydrogel composition, wherein the thermosensitive hydrogel composition comprises: i) a support phase, wherein the support phase comprises: 1) a primary component, wherein the primary component comprises a continuous or granular polymeric phase dissolved in an aqueous solution, further wherein the primary component is capable of Bingham plastic rheological behavior in the presence of an aqueous media; and 2) a secondary component, wherein the secondary component comprises a biocompatible thermally-desolubilizable polymer or co-polymer that exists in an extended form below a critical solution temperature (CST) that is is lower than the normal body temperature of the mammal and in a condensed form at or above the CST; b) depositing an embedded phase on top of the support phase at or below the CST of the biocompatible thermally-desolubilizable polymer or co-polymer of the support phase, wherein the embedded phase comprises at least one uncrosslinked polymer and further comprises a cell; and c) culturing the at least one cell, wherein during the culturing, the temperature of the composition is increased to at or above the CST of the biocompatible thermally-desolubilizable polymer or co-polymer of the support phase following deposition of the embedded phase.

In some aspects, the cell comprises a single cell. In some aspects, the cell comprises more than one cell. In some aspects, the cell comprise single cells, cell aggregates, or organoids. In some aspect, the cell comprises allogenic or autologous mesenchymal stem cells or induced pluripotent stem cells. In some aspects, the cell is derived from a plant, fungi, or algae. In some aspects, the cell is derived from a medicinal mushroom, a medicinal mycological culture, or a therapeutic fungi, such a shiitake mushroom. In some aspects, the cell is derived from an aquatic plant, such as, for instance, Spirulina algae and aloe vera. In some aspects, the cell is derived from a plant known for medicinal qualities such as, for instance, Ocimum basilicum, Curcuma longa, Zingiber officinale, and Vitis vinifera. In some aspects, during the culturing phase d) of the method, the cells are treated with soluble factors in the medium. In some aspects, such treatment can be used to induce at least one cellular process of the cultured cells. For instance, the cellular process can be related to, for example, nucleus pulposus, annulus fibrosus, hyaline cartilage, elastic cartilage, fibrocartilage, tendon, ligament, long bones, short bones, flat bone, irregular bones, cardiac tissue, or vascular tissue. In some aspects, the cell comprises a cell type that performs at least one function of the musculoskeletal or cardiovascular system. In some aspects, the cell type comprises nucleus pulposus, annulus fibrosus, hyaline cartilage, elastic cartilage, fibrocartilage, tendon, ligament, long bones, short bones, flat bone, irregular bones, cardiac tissue, or vascular tissue.

In some aspects, the temperature of the hydrogel composition comprising the embedded phase is decreased to below the CST during the culturing phase. In some aspects, the temperature of the hydrogel composition comprising the embedded phase is cycled between the temperature being greater than or equal to the CST and the temperature being less than the CST at least once during the culturing phase. In some aspects, the duration of the culturing phase is any user defined length of time for the culturing phase. In some aspects, the duration of the culture phase between about 1 hour and about 10,000 hours. In some aspects, the duration of the culturing phase is between about 1 day and about 100 days. In some aspects, the duration of the culturing phase is between about 1 day and about 50 days. In some aspects, the temperature is cycled between at least 1 time and at least 1,000 times during the culturing phase. In some aspects, the time period for each cycle is between about 15 minutes to about 5 hours. In some aspects, the temperature is cycled from about 1 time to about 1000 times, from about 1 time to about 500 times, from about 1 time to about 250 times, from about 1 time to about 200 times, from about 1 time to about 150 times, from about 1 time to about 100 times, from about 1 time to about 50 times, from about 1 time to about 40 times, from about 1 time to about 30 times, from about 1 time to about 20 times, from about 1 time to about 10 times, or from about 1 time to about 5 times during the culturing phase. In some aspects, In some aspects, the temperature is cycled from about 1 time to about 1000 times, from about 1 time to about 500 times, from about 1 time to about 250 times, from about 1 time to about 200 times, from about 1 time to about 150 times, from about 1 time to about 100 times, from about 1 time to about 50 times, from about 1 time to about 40 times, from about 1 time to about 30 times, from about 1 time to about 20 times, from about 1 time to about 10 times, or from about 1 time to about 5 times weekly. In some aspects, the temperature is cycled not more than 3 times per week of the culture phase. In some aspects, the duration of the temperature cycle below the CST is less than 30 minutes for each of 3 cooling periods within a one week time period. In some aspects, lowering the temperature below the CST promotes reversible de-adhesion of proteins and cells from the support phase. In some aspects, the temperature is cycled between 4° C. and 37° C. In some aspects, temperature is cycled between 25° C. and 37° C. In some aspects, increasing the temperature above the CST of the biocompatible thermally-desolubilizable polymer or co-polymer of the support phase following deposition of the embedded phase can cause the secondary component of the support phase to condense around the primary component. Such condensation, in some instances, can enhance overall long-term mechanical stability of the support phase during immersion in cell culture medium. Furthermore, in some instances, this transition can promote mechanical stabilization of the boundary between the support phase and the embedded phase, thereby aiding in maintaining geometry of aggregated cells and tissues within the compartment, or in maintaining the cells on top of the support phase. In some aspects, lowering the temperature below the CST of the biocompatible thermally-desolubilizable polymer or co-polymer of the support phase induces protein and cell release. In some aspects, cycling temperature across the CST inhibits maturation of focal adhesions and the formation of filopodium and lamellipodium extensions from the cells. In some aspects, cycling temperature across the CST inhibits cell motility through the support phase. In some aspects, anchorage-dependent cells can preferentially bind to one another during such temperature cycling, thereby promoting increased fusion of cells corresponding with the geometry of the embedded compartment in which the cells were placed. In some aspects, maintaining temperature of the culture system above the CST of the biocompatible thermally-desolubilizable polymer or co-polymer of the support phase can promote adsorption of proteins such as fibronectin from the culture to the support phase, the gradual maturation of focal adhesions to the support phase, and the spreading and migration of cells throughout the support phase.

In some aspects, such temporal control over cell adhesion, such as can be promoted by temperature cycling, can allow for a user to manipulate cell approximation and morphology. For instance, persistent regular disruptions to focal adhesions over the culture period via cycling across the CST can promote cell roundness and maturation of cell-cell adhesions which can be favorable for efficient cellular communication and chondrogenic differentiation. Alternatively, cells may be persistently disrupted from focal adhesions early in the culture period and subsequently allowed to adhere and migrate through the support phase at a later stage. In general, cytoskeletal adaptation to adhesive cues is not transient, meaning, cellular memory from temporarily applied adhesive cues persists and impacts global cell behavior for culture duration. As such, programs can be optimized according to the needs of the particular tissue engineering application and implemented by simple and cell-friendly heating and cooling at cooling cycles. Furthermore, geometric control over the embedded regions of high cell density can be coupled with dynamic temporal control over adhesion cues to maintain cells in desired patterns over the culture period and/or condense them further. Embedding and fusing cells longitudinally within embedded channels, following any spatial pattern, can allow for mimicking of the anisotropic cell distributions of musculoskeletal tissue. In contrast, cell aggregates typically are cultured in a purely spherical geometry. By using the embedded compartment to constrain fusion of the embedded cells longitudinally, larger numbers of cells can be aggregated in a configuration that bypasses the diffusion limitations of the radially-expanding spherical geometries. In some aspects, cell aggregates within the embedding medium are extruded on top of the embedded phase, and allowed to interact with its exposed surface area for the implementation of the temperature cycles.

### Secretome Collection

In some aspects, a secretome expressed by the cell is collected. In some aspects, the conditioned medium above the exosomes is collected. For instance, the conditioned medium can be collected following at least about 2 days, at least about 3 days, at least about 4 days, at least about 5 days, at least about 6 days, or at least about 7 days. In some aspects, the conditioned medium is pooled from more than one hydrogel. In some aspects, the conditioned medium is pooled from 2, 3, 4, 5, 6, 7, 8, 9, 10, 25, 50, 75, or 100 hydrogels, and all integers therebetween. In some aspects, exosomes are isolated from the condition medium by ultracentrifugation. In some aspects, the supernatant is collected from such ultracentrifugation, and can in some instances be collected after more than one ultracentrifugation step. In some aspects, the cargo of the exosomes can be characterized using gene, nucleic acid, and/or protein-based analysis, such as including, but not limited to, microRNA array, western blot, global proteomics analysis, gene ontology analysis, real-time PCR (RT-PCR), and/or nucleic acid sequencing. In some aspects, the secretome is analyzed to generate a cytokine profile.

As such, in another aspect, the present disclosure generally relates to a method for culturing a cell, wherein the method comprises a) providing a thermosensitive hydrogel composition comprising a support phase and an embedded phase, such as a thermosensitive hydrogel composition comprising a support phase and an embedded phase as described herein; b) depositing an embedded phase into the support phase at or below a CST of the support phase, wherein the embedded phase comprises at least one uncrosslinked polymer and further comprises a cell; c) culturing the at least one cell, wherein during the culturing, the temperature of the composition is increased to at or above the CST of the support phase following deposition of the embedded phase; and d) collecting the secretome expressed by the cell. In one aspect, the present disclosure generally relates to a method for culturing a cell, wherein the method comprises a) providing a thermosensitive hydrogel composition comprising a support phase and an embedded phase, such as a thermosensitive hydrogel composition comprising a support phase and an embedded phase as described herein; b) depositing an embedded phase onto the support phase at or below a CST of the support phase, wherein the embedded phase comprises at least one uncrosslinked polymer and further comprises a cell; c) culturing the at least one cell, wherein during the culturing, the temperature of the composition is increased to at or above the CST of the support phase following deposition of the embedded phase; and d) collecting the secretome expressed by the cell. In another aspect, the present disclosure generally relates to a method for culturing a cell, wherein the method comprises: a) providing a thermosensitive hydrogel composition, wherein the thermosensitive hydrogel composition comprises: i) a support phase, wherein the support phase comprises: 1) a primary component, wherein the primary component comprises a continuous or granular polymeric phase dissolved in an aqueous solution, further wherein the primary component is capable of Bingham plastic rheological behavior in the presence of an aqueous media; and 2) a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b) depositing an embedded phase into the support phase at or below the CST of the biocompatible thermally-desolubilizable polymer or co-polymer of the support phase, wherein the embedded phase comprises at least one uncrosslinked polymer and further comprises a cell; c) culturing the at least one cell, wherein during the culturing, the temperature of the composition is increased to at or above the CST of the biocompatible thermally-desolubilizable polymer or co-polymer of the support phase following deposition of the embedded phase; and d) collecting the secretome expressed by the cell. In another aspect, the present disclosure generally relates to a method for culturing a cell, wherein the method comprises: a) providing a thermosensitive hydrogel composition, wherein the thermosensitive hydrogel composition comprises: i) a support phase, wherein the support phase comprises: 1) a primary component, wherein the primary component comprises a continuous or granular polymeric phase dissolved in an aqueous solution, further wherein the primary component is capable of Bingham plastic rheological behavior in the presence of an aqueous media; and 2) a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b) depositing an embedded phase onto the support phase at or below the CST of the biocompatible thermally-desolubilizable polymer or co-polymer of the support phase, wherein the embedded phase comprises at least one uncrosslinked polymer and further comprises a cell; c) culturing the at least one cell, wherein during the culturing, the temperature of the composition is increased to at or above the CST of the biocompatible thermally-desolubilizable polymer or co-polymer of the support phase following deposition of the embedded phase; and d) collecting the secretome expressed by the cell

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Preparation of an Exemplary Thermally Sensitive Hydrogel Composition

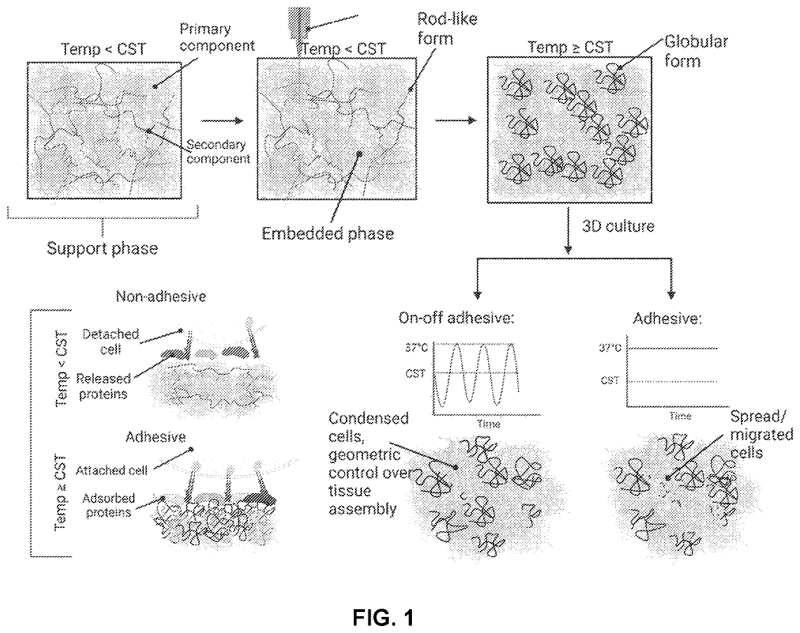

The present example relates to preparations of exemplary thermally sensitive hydrogel compositions in accordance with the present disclosure. For instance, FIG. 1 presents a flowchart summarizing an example of a hydrogel composition and an example of a method of application. Referring to FIG. 1, a support phase was homogenously comprised of primary and secondary components in an aqueous solvent and exhibited critical solution temperature (CST) behavior, where below the CST (temp<CST) the support phase was non-adhesive and at or above the CST (temp≥CST) the support phase was adhesive. A cell-laden soft material was dispensed by an extrusion tip into the support phase to create an embedded phase while temperature was maintained below the CST. Post deposition of the embedded phase, the construct was heated (temp≥CST) to induce molecular collapse of the secondary component of the support phase into a globular conformation which increased elastic modulus and overall mechanical stability. During a period of long-term 3D culture thereafter, temperature could be cycled temporally below the CST or maintained static at the CST to promote either aggregation or spreading/migration, of the embedded cells, respectively.

An exemplary composition of the support phase was prepared as follows. Chondroitin 4-sulfate sodium salt (CS, from bovine trachea, Sigma, St. Louis, USA) was functionalized with methacrylate groups using methacrylic anhydride (MA), in a molar ratio of 25:1 (MA:CS). N-isopropylacrylamide (NIPAAm) monomer (Acros Organics, Geel, Belgium) was polymerized in the presence of methacrylate functionalized chondroitin sulfate using a molar ratio of NIPAAm:CS of 2500:1 to generate the pNIPAAm-CS graft copolymer. The free radical reaction was initiated by ammonium persulfate (0.8% of total monomer content) and accelerated by tetramethylethylenediamine (8.0% of total monomer content). After synthesis and purification, the graft copolymer was dissolved in deionized water at a concentration of 3% (w/v) pNIPAAm-CS. Next, polyacrylic acid microgels (Carbopol® 940, Acros Organics Geel, Belgium) and gelatin (from porcine skin, 175 g Bloom, type A, Sigma, St. Louis, USA) were co-dissolved with the pNIPAAM-CS at concentrations of 0.8 and 1% (w/v), respectively. After homogenous dissolution of all components, the pH was adjusted to 7.4 with 50% sodium hydroxide (NaOH) and the hydrogel was stored at 4° C. until testing.

### Example 2: Rheological Testing of Exemplary Thermally Sensitive Hydrogel Compositions

Rheological testing of the support phase was conducted with an Anton Paar MCR-302 rheometer (Anton Paar GmbH, Austria). All measurements were taken with a gap size of 0.5 mm and a parallel plate with a diameter of 25 mm. Unless stated otherwise, the rheological properties were assessed at a temperature of 25° C., with a frequency of 1 Hz and with n=3 samples per group. To prevent sample drying during the measurements, silicone oil was applied around the plate to seal the hydrogel. Aqueous PAA 0.8% (w/v) served as control group for all tests as it is a standard freeform printing medium. For all quantitative comparisons, the results were tested for normality and lognormality. Subsequently, normally distributed results were evaluated for their statistical significance with one- or two-way analysis of variance (ANOVA), respectively. Multiple comparisons were analyzed post-hoc with a Tukey Honest Significance Difference Test. P-values p<0.05 were considered significant, significance levels are as follows: * p<0.05, ** p<0.01, *** p<0.001 and **** p<0.0001.

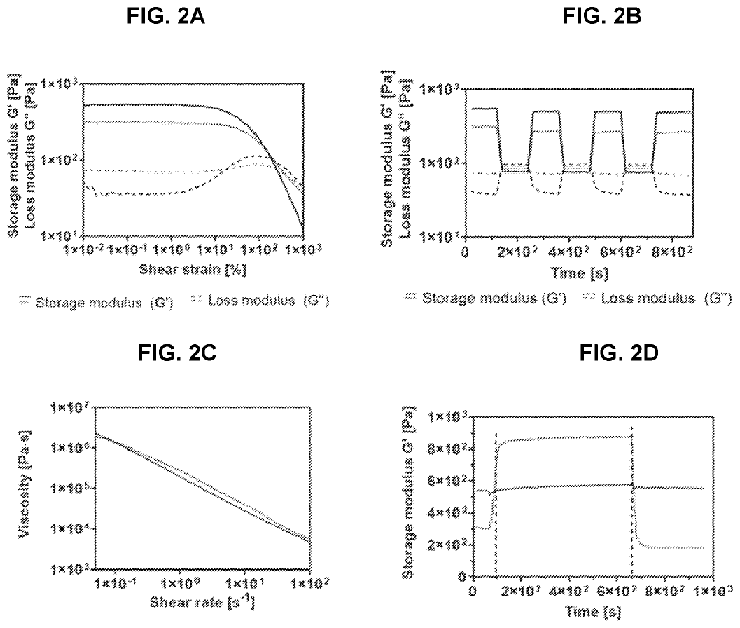

Amplitude sweep tests (FIG. 2A) were performed with oscillatory strain in the range of 0.01% and 1000% strain applied to the exemplary support phase. Similar to the behavior of 0.8% PAA control (black), the exemplary support phase was solid-like at rest and fluidized when exposed a threshold yield stress. The yield stress was taken at the point of the curves cross-over (storage modulus, G′=loss modulus, G″). The 0.8% PAA controlled exhibited a flow stress of 277±4 Pa compared to 366±43 Pa for the exemplary support phase formulation (p<0.0028). The confirmed presence of a yield stress for the exemplary support phase demonstrated its suitability for providing mechanical support to spatially embedded materials dispensed by an extrusion.

In an oscillatory step strain test (FIG. 2B), G′ and G″ were measured while the strain was alternated between a low strain, set to 1%, and a high strain, set to 250%, over the course of seven intervals. Each interval had a duration of 120 s, except the 7th which lasted 160 s at 1% strain. The step strain test revealed that the exemplary support phase (grey, FIG. 2B) exhibited a similar capacity for shear-recovery to the 0.8% PAA control (black, FIG. 2B). This self-recovery capacity was maintained over all tested cycles between low and high strain and was indicative that the exemplary support phase can self-heal quickly post-needle translation.

A rotational viscosity test was performed to demonstrate the comparable shear-thinning properties of the exemplary support phase (grey, FIG. 2C) compared to the 0.8% PAA control (black, FIG. 2C). The viscosity η was measured as the shear rate was increased over the range of 0.01 s⁻¹ to 100 s⁻¹ The decreasing viscosity with increasing shear rate demonstrated that the exemplary support phase could facilitate nozzle translation through it, similar to the control.

An oscillatory temperature ramp test with 1% constant strain was used to evaluate viscoelastic properties as a function of temperature (FIG. 2D). The test was divided into three intervals, the first of which was a 60 s measurement at 25° C. After this, the temperature was increased to 37° C. in the second interval for 600 s. In the third interval, the temperature was reduced to 25° C. and the measurements were taken for 300 s. The storage modulus at 37° C. of the exemplary support phase significantly increased from 307±58 Pa at 25° C. to 868±33 Pa (grey, FIG. 2D, p<0.0001). By comparison, the 0.8% PAA control exhibited no change in storage modulus with temperature (Black, FIG. 2D). The results demonstrated a temperature-triggered increase in G′ (i.e., thermally-triggered gelation) for the experimental group only.

### Example 3: Print Fidelity Testing of Exemplary Thermally Sensitive Hydrogel Compositions

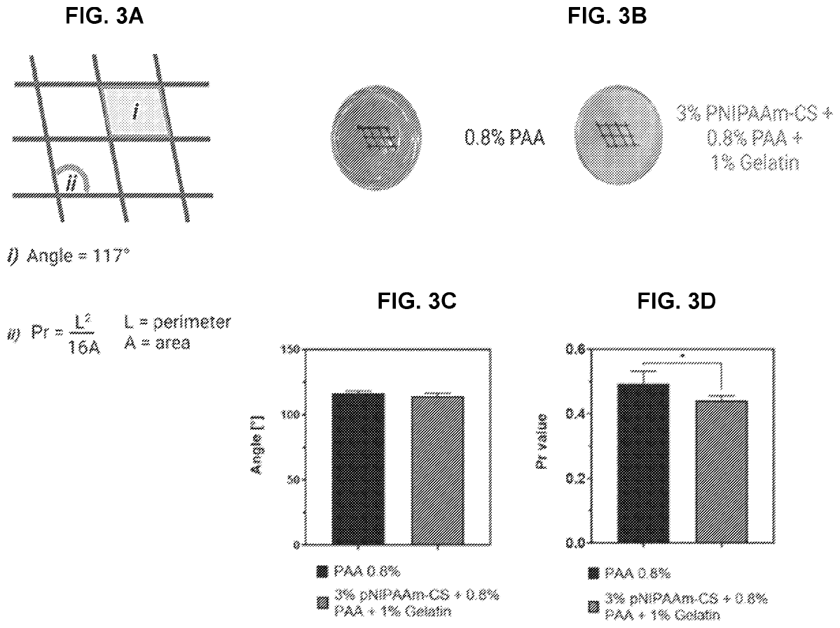

Extrusion of the soft embedded material within the gel composite was performed with a 3D Discovery™ Printer and BioCAD software (version 1.1-12) from RegenHu Ltd. (Villaz-St-Pierre, Switzerland) using a PTFE-lined needle with 0.3 mm inner diameter (Nordson EFD, Westlake, USA). For print fidelity studies, the embedded phase was comprised of an acellular, 6% (w/v) gelatin solution which was extruded into the exemplary support phase. A free-floating horizontal grid geometry to evaluate print fidelity was used (FIG. 3A). The 6% gelatin solution was deposited at a height of 2 mm above the bottom inside the gel composite at a velocity of 0.5 mm/s, and with a pressure of 0.5-1.5 bar. For visualization of the deposited pattern, the 6% gelatin was supplemented with 0.1% (w/v) Coomassie Blue (FIG. 3B). Digital photos of the printed grid were analyzed for angle between the strands and for Pr value (Equation 2), and they were compared to the values of the originally designed structure. A total of n=10 measurements per group were made of both angle and Pr value with ImageJ (version 1.53c, National Institutes of Health (NIH), MD, USA) (FIG. 3C).

Pr=L²/16A (Equation 2)

When analyzing angle accuracy, the angle measurements for the exemplary support phase were not significantly different than the 0.8% PAA control, indicating comparable freeform printing accuracy to standard medium (FIG. 3C). The exemplary support phase exhibited a slight but statistically significant lower Pr value compared to the 0.8% PAA control (p=0.022, FIG. 3D).

### Example 4: Small Angle X-ray Scattering Analysis of Exemplary Thermally Sensitive Hydrogel Compositions

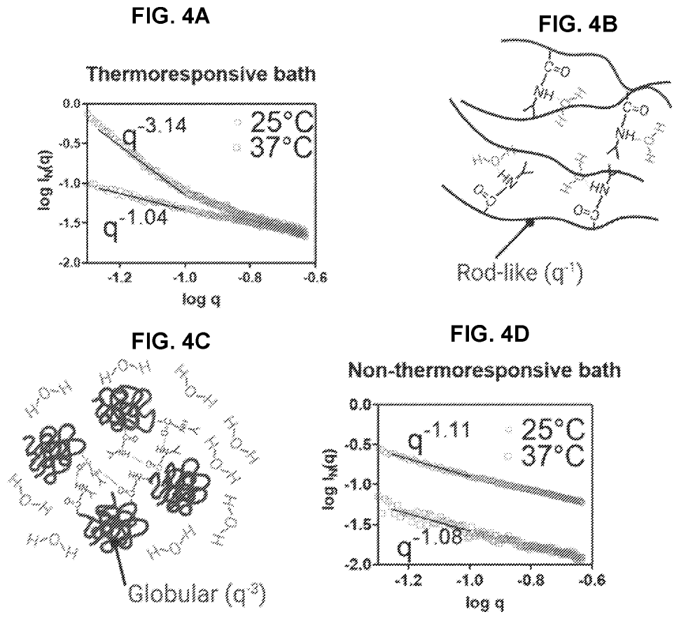

Small angle x-ray scattering (SAXS) was used to characterize the molecular conformation of an exemplary temperature-sensitive support phase. These data were collected at Diamond Light Source (Didcot, UK, SM29767). Dilute support bath samples (0.3 w/v % pNIPAAM, 0.08 w/v % CP and 0.1 w/v % gelatin) were collected and measured at 25 or 37° C., for 21 frames with 1 s exposure each. The sample-to-detector distance was 3.712 m, operating at 13.018 keV with a 3.7 m camera length. From the SAXS measurements, the Guinier slope was extracted defining fractal dimension (qn) to provide information on the dimensional structure of the support bath particles. The Guinier slope of n=−1.04 was extracted at 25° C. (FIG. 4A, circles) suggesting that the support phase consisted of rod-like polymeric chains with weak physical entanglement, as for instance presented in the schematic of FIG. 4B. Thus, at this temperature, the blended network was fully hydrated. As temperature was increased to 37° C., n shifted to a value of −3.14 (FIG. 4A, squares), indicating that the network transitioned to a collapsed entangled globule with a rough surface, as for instance presented in the schematic of FIG. 4C. For the non-thermoresponsive bath without the pNIPAAM, values of n=−1.11 and n=−1.08 were obtained at 25 and 37° C., respectively (FIG. 4D), which thereby indicated a rod-like structure (FIG. 4B) and that the temperature responsiveness of the bath is attributable to the pNIPAAM component.

### Example 5: Cell Culture Studies

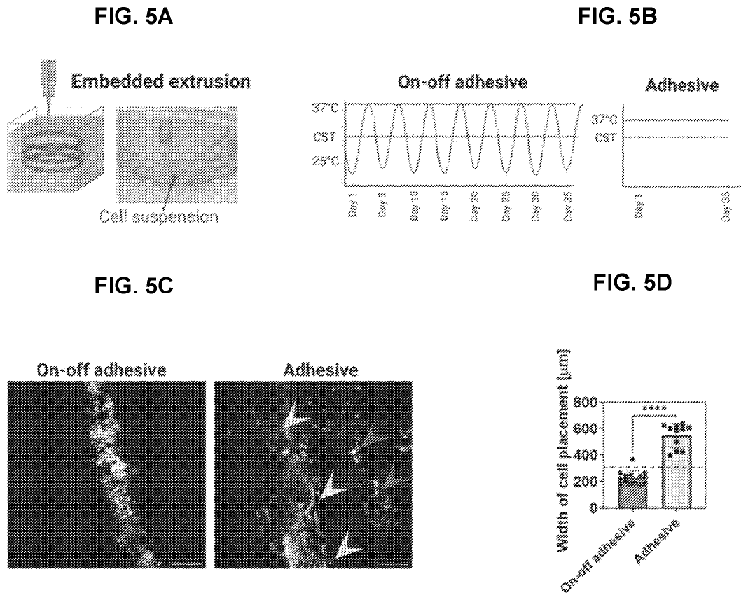

For the cell culture studies described below, 4 million human bone marrow derived mesenchymal stem cells (MSCs) were suspended in single cell form per milliliter of 6% gelatin dissolved in chondropermissive cell culture medium. This single cell suspension was 3D printed through a PTFE-lined needle with 0.3 mm inner diameter into an exemplary support phase. The extrusion pattern consisted of three circles (FIG. 5A) with a radius of 5 mm, stacked in Z-direction with 1 mm spacing, and was deposited at a starting height of 2 mm inside the hydrogel. Printing parameters were as follows: 23° C. ambient temperature, 16% relative humidity, 0.2-0.8 bar printing pressure, 25° C. bioink temperature, and 0.5 mm/s deposition velocity. The patterned cells were cultured for 5 weeks in chondropermissive medium under static adhesive conditions (37° C.), or on-off adhesive conditions (cycling between 37° C. and 25° C. for 15 min every 5 days, FIG. 5B). The chondropermissive medium was composed of Dulbecco's modified Eagle medium 4.5 g/L glucose (Gibco, Carlsbad, USA), sodium pyruvate 0.11 g/L (Sigma-Aldrich, Buchs, Switzerland), L-ascorbic acid 2-phosphate sesquimagnesium salt hydrate (50 μg/mL. Sigma-Aldrich, Buchs, Switzerland), dexamethasone (100 nM. Sigma-Aldrich, Buchs, Switzerland), insulin transferrin and selenium 1% (Cyangen, Guangzhou, China), and Non-essential amino acids 1% (Gibco, Carlsbad, USA). Cell morphology was examined using phalloidin and DAPI counterstain. Semiquantitative analysis of the width of the cell placement was done with ImageJ by plotting intensity as a function of x position. N=10 photos were evaluated across 3 gels per condition. Confocal images taken at week 5 of culture (FIG. 5C) revealed that BM-hMSCs cultured in on-off adhesive conditions presented in an aggregated channel pattern with round cell nuclei. In contrast, the BM-hMSCs that were cultured in static conditions displayed a tendency for elongation (FIG. 5C, white arrows) and migration into the support phase (FIG. 5C, grey arrows). The width of the cell placement within the ringed channel pattern for the on-off adhesive conditions was 229.64±52.0 μm versus 548±92.1 μm for adhesive conditions (FIG. 5D, p<0.0001). It was observed that cells extruded through a 300 μm inner diameter needle and subjected to on-off adhesive treatment during the culture period resulted in their increased condensation towards each other, thereby demonstrating that periodic cooling across the CST of the support phase promotes cell condensation.

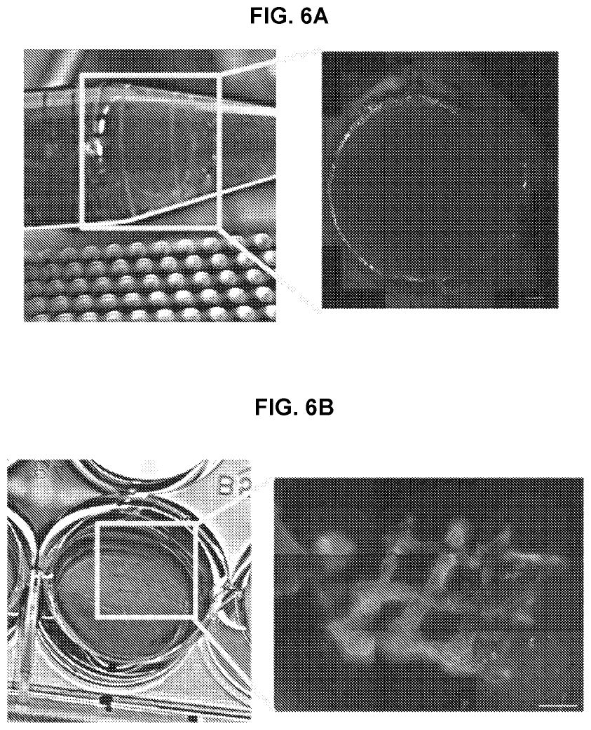

The effects of a long-term culture period of 35 days while performing on-off adhesive conditions were analyzed. After the long-term culture period of 35 days, on-off adhesive conditions produced compact and mechanically competent cell and tissue structures of directed and anisotropic geometries (FIG. 6A-FIG. 6B). For instance, referring to FIG. 6A-FIG. 6B, macroscopic and confocal imaging of tissue structures assembled by cells patterned within the exemplary hydrogel composition were performed. BM-MSCs extruded in a ring or necklace-like configuration and cultured in chondropermissive medium for 35 days under on-off adhesive conditions (cooled to 25° C. for 15 min every 5 days) produced a mechanically competent structure with geometry matching the embedded compartment generated within the support phase (FIG. 6A, left panel). The cell/tissue structure stayed cohesive after dilution in cell culture medium (FIG. 6A, inset). Similarly, L929 murine fibroblasts were extruded in a grid-like geometry, cultured in Low glucose Dulbecco's Modified Eagle Medium with 10% serum, and condensed by the on-off adhesive conditions applied over the culture period to produce a mechanically stable cell/tissue structure with geometry matching the embedded phase (FIG. 6B).

### Example 6: Aggregate Shaping

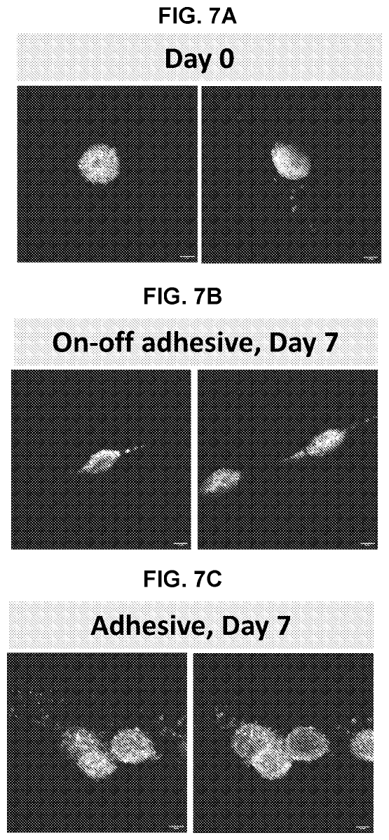

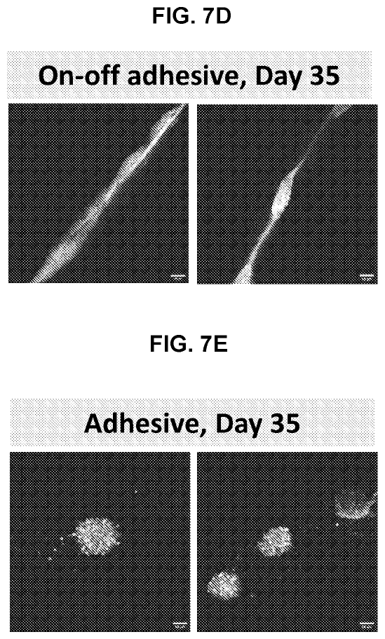

Human bone marrow derived mesenchymal stem cells (MSCs, P4) were formed into spherical aggregates by overnight incubation in chondropermissive medium inside AggreWell™ 400 6-well plates. The aggregates, comprised 250,000 cells each, were suspended in 6% porcine gelatin bioink at a density of 4×10⁶ total cells/mL. The aggregate suspension was microextruded through a 300 μm (inner diameter) needle into the support bath and cultured in chondrogenic medium composed of high glucose DMEM (4.5 μg/ml D-Glucose), sodium pyruvate (0.11 μg/ml) and sodium bicarbonate (3.7 μg/ml) with 1% Pen/Strep, L-ascorbic acid 2 phosphate (50 μg/ml), 1% ITS, non-essential amino acids (5 mg/ml), dexamethasone (0.04 ng/ml) and 10 ng/mL TGF-β1. Constructs (n=3 per group per time point) were either maintained under static temperature at 37° C. for 35 days (adhesive conditions) or on-off adhesive conditions by cooling to 25° C. for 10 min at days 1, 3, 5, 10, 15, 20, 25, 30, and 35. At days 0, 7, and 35, constructs were stained DAPI to highlight cell nuclei and TRITC-conjugated phalloidin to visualize cell cytoskeleton (FIG. 7A-FIG. 7D). Imaging results show that at day 0, immediately after embedding, the cell aggregates were round with diameter approx. 150 μm (FIG. 7A). After 7 days of culture under on-off adhesive conditions, the aggregates started to take on an elongated oval appearance (FIG. 7B). In contrast, aggregates were cultured under adhesive conditions remained spherical and appeared to possess looser cell packing (FIG. 7C). After 35 days of culture under on-off adhesive conditions, cell aggregates adopted appreciably more oblong morphology and had begun fusing together (FIG. 7D). Under adhesive conditions, the aggregates remained spherical and no instances of fusion were detected (FIG. 7E). It was observed that on-off adhesive conditions resulted in gradual elongation of the aggregates to significantly more oblong morphology and resulted in some fusion by day 35. Aggregates cultured under adhesive conditions generally retained round morphology and demonstrated no instances of fusion across the 35-day study. This disclosure demonstrates the elongation of a musculoskeletal cell aggregate to an oblong morphology in a 3D matrix. On-off adhesive conditions also appeared to promote aggregate proximity and therefore fusion.

### Example 7: Rheological Testing of Additional Thermally Sensitive Hydrogel Compositions

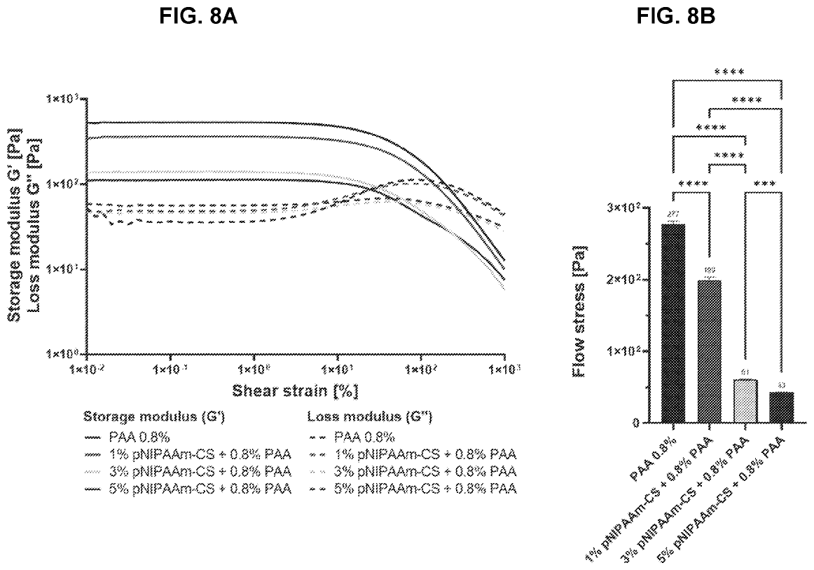

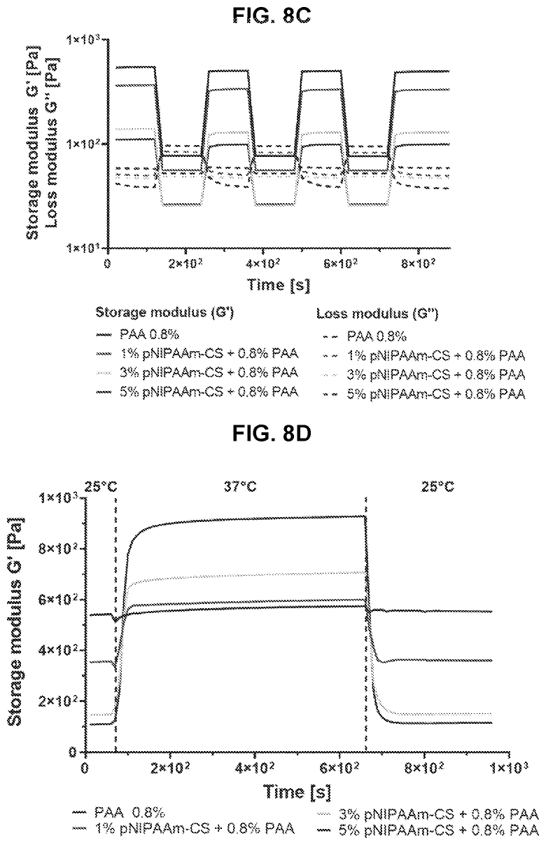

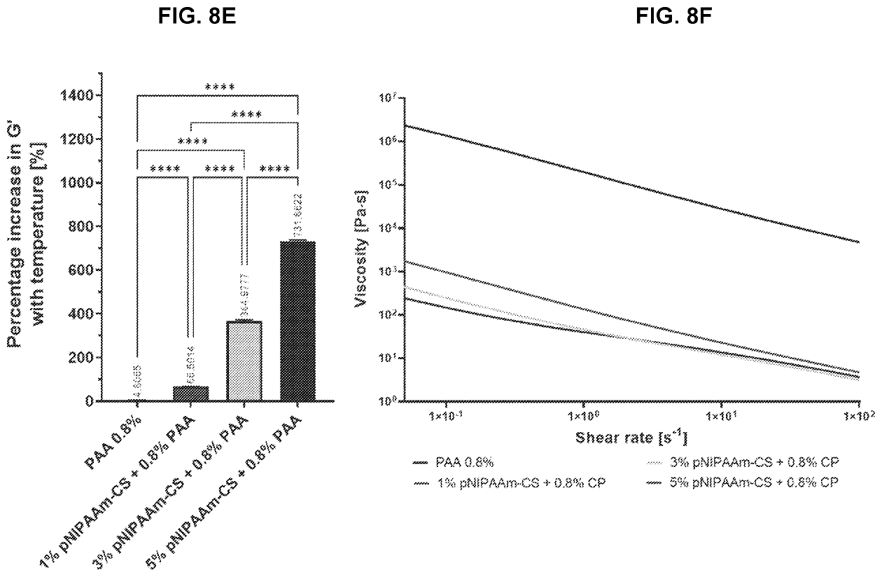

Additional compositions of thermally sensitive hydrogel compositions (1, 3 or 5% pNIPAAm-CS+0.8% PAA) were tested for rheology properties. Amplitude sweep tests were performed with oscillatory strain in the range of 0.01% and 1000% strain. The flow stress of the formulations was calculated at the point of the curves cross-over (G′=G″) using the integrated analysis tool of the RheoCompass™ software (version 1.26.22. Anton Paar GmbH, Austria). Similar to the behavior of the 0.8% PAA control, the results shown in FIG. 8A indicate that 1, 3 and 5% pNIPAAm-CS+0.8% PAA were solid-like at rest (<1% strain, within LVR) and fluidized when exposed to a certain formulation-specific yield stress. The PAA 0.8% control had the highest flow stress (277±4 Pa) at 25° C., compared to 199±4 Pa, 61±0 Pa, and 43±0 Pa for 1%, 3% and 5% pNIPAAm-CS+0.8% PAA, with all pair-wise comparisons being statistically significant (p<0.001, FIG. 8B). Overall, solid like behavior of the gel composite suggested their suitability for providing mechanical support to spatially embedded soft materials dispensed by an extrusion tip.

In an oscillatory step strain test, G′ and G″ were measured while the strain was alternated between a low strain, set to 1%, and a high strain, set to 250%, over the course of seven intervals. The step strain test revealed that 1, 3 and 5% pNIPAAm-CS+0.8% PAA have a potential for shear-recovery similar to the 0.8% PAA control (FIG. 8C). This self-recovery capacity was maintained over at least three cycles between low and high strain with recovery rates of 92±1%, 91±1%, 92±0% and 89±0% for PAA 0.8%, and 1%, 3% and 5% pNIPAAm-CS+0.8% PAA, which indicated suitability of the gel composites to accommodate translation of a nozzle or aspiration tip for extrusion.

An oscillatory temperature ramp test with 1% constant strain (within linear viscoelastic region (LVR) as established by primary amplitude test) was used to evaluate viscoelastic properties as a function of temperature (FIG. 8D). The test was divided into three intervals, the first of which was a 60 s measurement at 25° C. After this, the temperature was increased to 37° C. in the second interval for 600 s. In the third interval, the temperature was reduced to 25° C. and the measurements were taken for 300 s. All pNIPAAm-CS+PAA composite hydrogels showed a reversible temperature-responsiveness and a significant increase in G′ of 67%, 365%, and 732% for 1%, 3% and 5% pNIPAAm-CS+0.8% CP, respectively, upon a temperature increase from 25° C. to 37° C. (FIG. 8E). PAA control exhibited only a slight increase in G′ with temperature (FIG. 8E, p<0.0001).

A rotational viscosity test was done to investigate the shear-thinning properties of the hydrogels (FIG. 8F). The viscosity η was measured, as the shear rate was increased over the range of 0.01 s-1 to 100 s-1. The shear-thinning behaviour of all hydrogel formulations was confirmed with a flow curve, which further supported the notion that the gel composite formulations are suitable for supporting nozzle translation and extrusion of soft materials within its structure.

Overall, these results illustrated that the primary phase can be combined with the secondary component in a broad range of concentrations and still retain Bingham plastic rheological properties allowing freeform extrusion of the embedded soft material.

### Example 8: Cell Secretome Collection

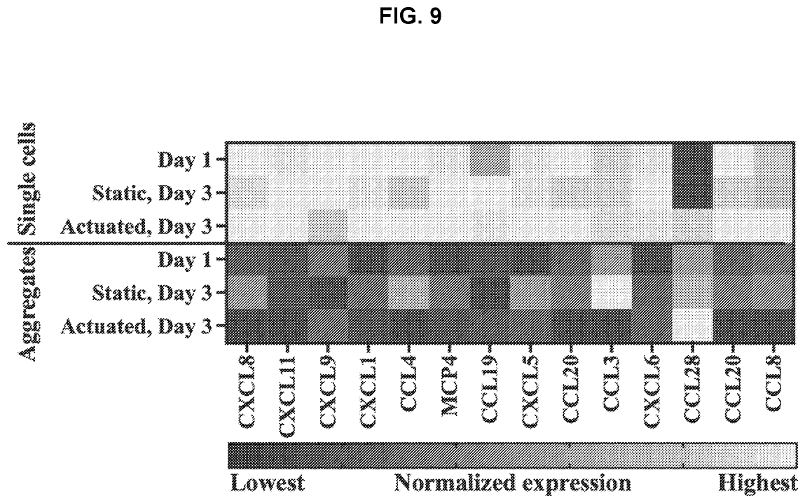

In the present example, the procedure for cell secretome collection is described. BM-MSCs are prepared as a single-cell suspension as described in Example 5, or in aggregate form as described in Example 6, and are cultured for a minimum of 35 days. The conditioned medium above the gel samples is collected periodically, following a minimum of 2 days of culture. Conditioned medium is collected periodically and profiled by high-multiplex immunoassay. See, for instance, Example 9 and FIG. 9. The conditioned medium undergoes exosome isolation by ultracentrifugation. The supernatant is collected after multiple centrifugation steps (for instance, 300 xg for 10 minutes at 4° C., 2000 xg for 20 minutes at 4° C., 10,000 xg for 20 minutes at 4° C., and 100,000 xg for 60 mins at 4° C.). The resulting pellet is washed in PBS, and ultracentrifuged at 100,000 xg for 60 mins at 4° C., resuspended in PBS and analyzed. The size, morphology, and count of the exosomes is determined with fluorescent nanoparticle tracking (FNT) and transmission electron microscopy (TEM). The cargo of the exosomes is characterized using genomic and proteomic analyses (such as, for instance, microRNA array, Western blot, gene ontology analysis, real-time PCR (RT-PCR)). On-off adhesive conditions, which promote single cell condensation, aggregate self-stretching, and aggregate fusion, are anticipated to alter global expression of signaling molecules especially related to tissue remodeling, cellular differentiation, inflammation and spatial organization of aligned musculoskeletal tissues.

### Example 9: Cell Secretome Collection and Analysis

In the present example, cell secretome was collected and analyzed. In particular, a cytokine profile was generated and analyzed using a high-multiplex immunoassay (Olink Proteomics Inc., Boston. MA).

Cytokines were expressed by 4 million cells/mL of bone marrow-derived MSCs within the support phase as either single-cell suspensions or spheroidal aggregates, and cultured under static adhesive or on-off adhesive conditions. The samples were cultured for a total of 3 days. On day 1 post-printing, the on-off adhesive samples experienced a single 15-minute cooling period at 25° C., while being maintained at 37° C. otherwise. Static adhesive samples were kept at 37° C. throughout the entire culture period. Conditioned medium samples were collected at day 0 (immediately post-printing) and day 3 for both experimental groups. The conditioned medium was pooled from n=3 hydrogels for each experimental group.

Referring now to FIG. 9, the results displayed in FIG. 9 present intensity values for each cytokine normalized to blank media. The color scale depicted in FIG. 9 illustrates cytokine concentrations, with purple indicating the lowest levels, green indicating the 50th percentile, and yellow indicating the highest concentration for each cytokine analyzed. The results indicated that culturing under on-off adhesive conditions as opposed to static adhesive conditions did not significantly alter the immunomodulatory expression profile of MSCs, whether in single-cell or aggregated form. However, it was observed that aggregated cells exhibited decreased expression levels of chemotactic chemokines compared to cells in single-cell format. Without wishing to be bound by theory, it is postulated that this distinction could be attributed to the focal point of migratory activities within aggregates, primarily involving leading-edge cells, while central cells tend to remain stationary. Without wishing to be bound by theory, it is postulated that the enhancement in cytokine expression for single cells could result from the single-cell form facilitating more efficient communication throughout the entire cell population, thereby promoting sustained interconnectedness and proximity.

### Enumerated Embodiments

In some aspects, the present invention is directed to the following non-limiting embodiments:

Embodiment 1: A thermosensitive hydrogel composition comprising: a. a support phase, wherein the support phase comprises: i. a primary component comprising a continuous or granular polymeric phase dissolved in an aqueous solution, further wherein the primary component is capable of Bingham plastic rheological behavior in the presence of an aqueous media; ii. a secondary component comprising a biocompatible thermally-desolubilizable polymer or co-polymer that exists in an extended form below a critical solution temperature (CST) that is lower than the normal body temperature of a mammal and in a condensed form at or above the CST; and b. an embedded phase, wherein the embedded phase comprises at least one uncrosslinked polymer and further comprises at least one cell, wherein the embedded phase is capable of extrusion into the support phase at or below the CST of the support phase.

Embodiment 2: The composition of embodiment 1, wherein the support phase comprises a homogenous mixture of primary and secondary components dissolved in aqueous solution below the CST of the secondary component.

Embodiment 3: The composition of any one of the foregoing embodiments, wherein the continuous or granular polymeric phase of the support phase comprises a biocompatible polymeric material.

Embodiment 4: The composition of any one of the foregoing embodiments, wherein the biocompatible thermally-desolubilizable polymer or co-polymer is in a wetted and/or rod-like state below the CST.

Embodiment 5: The composition of any one of the foregoing embodiments, wherein the secondary component is capable of releasing or being resistant to cell and protein adhesion below the CST.

Embodiment 6: The composition of any one of the foregoing embodiments, wherein the biocompatible thermally-desolubilizable polymer or co-polymer is in a globular, hydrophobic, gelated form at or above the CST.

Embodiment 7: The composition of any one of the foregoing embodiments, wherein the secondary component is capable of being adhesive for cells and proteins at or above the CST.

Embodiment 8: The composition of any one of the foregoing embodiments, wherein the at least one uncrosslinked polymer of the embedded phase comprises a biocompatible polymer.

Embodiment 9: The composition of any one of the foregoing embodiments, wherein the at least one uncrosslinked polymer of the embedded phase is dissolved in growth media.

Embodiment 10: The composition of any one of the foregoing embodiments, wherein the embedded phase is distributed across the support phase in spatially segregated compartments.

Embodiment 11: The composition of embodiment 10, wherein the spatially segregated compartments are spherical or cylindrical.

Embodiment 12: The composition of embodiment 10 or embodiment 11, wherein the spatially segregated compartments are continuous.

Embodiment 13: The composition of embodiment 10 or embodiment 11, wherein the spatially segregated compartments are discontinuous.

Embodiment 14: The composition of any one of embodiments 10-13, wherein the spatially defined compartments are anisotropic.

Embodiment 15: The composition of any one of the foregoing embodiments, wherein the overall concentration of the continuous or granular polymeric phase dissolved in an aqueous solution of the support phase is from about 0.10% to about 90%.

Embodiment 16: The composition of any one of the foregoing embodiments, wherein the continuous or granular polymeric phase dissolved in an aqueous solution is selected from the group consisting of poly(ethylene oxides), poly(propylene oxides), copolymers of PEO and polylactic acid (PLA), polyvinyl alcohol, celluloses, agar, agarose, chitosan, alginate, collagen, celluloses, polyacrylic acid, hyaluronates, keratins, and decellularized tissue components from any one of intervertebral disc, tendon, ligaments, cartilage, bone, cardiac tissues, and vascular tissues

Embodiment 17: The composition of any one of the foregoing embodiments, wherein the secondary component of the support phase comprises at least one material that is distinct from the primary component of the support phase.

Embodiment 18: The composition of any one of the foregoing embodiments, wherein the overall concentration of the secondary component is about 1% to about 25%.

Embodiment 19: The composition of any one of the foregoing embodiments, wherein the secondary component comprises at least one polymer selected from the group consisting of poly(N-isopropyl acrylamides) (PNIPAAm), poly(N,N-diethylacrylamide), poly(N-vinylcaprolactam), poly(2-oxazolines), poly(2-dimethylamino)ethyl methacrylate), and poloxamers ((poly(ethylene oxide) (PEO)-b-poly(propylene oxide)-b-PEO).

Embodiment 20: The composition of any one of the foregoing embodiments, wherein the biocompatible thermally-desolubilizable polymer or co-polymer of the secondar component is covalently linked to a polymer selected from the group consisting of PEO, polylactic-coglycolic acid, alginate, hyaluronic acid, gelatin, collagen, chondroitin sulfate, and decellularized extracellular matrix components.

Embodiment 21: The composition of embodiment 20, wherein the decellularized extracellular matrix components are derived from tendon, ligament, bone, cartilage, intervertebral disc, vascular tissues, and/or cardiac tissues.

Embodiment 22: The composition of any one of the foregoing embodiments, wherein the secondary component comprises PNIPAAm.

Embodiment 23: The composition of embodiment 22, wherein the PNIPAAm is covalently linked to chondroitin sulfate.

Embodiment 24: The composition of any one of the foregoing embodiments, wherein the secondary component comprises PNIPAAm at an overall concentration of about 1% to about 25%.

Embodiment 25: The composition of any one of the foregoing embodiments, wherein the support phase comprises a yield point of 50-1000 Pa below the normal body temperature of the mammal.

Embodiment 26: The composition of any one of the foregoing embodiments, wherein the support phase exhibits at least 25% greater elastic modulus above the CST of the secondary component.

Embodiment 27: The composition of any one of the foregoing embodiments, wherein the embedded phase comprises at least one of gelatin, collagen, chitosan, alginate, collagen, cellulose, modified celluloses, and soluble decellularized tissue components.

Embodiment 28: The composition of embodiment 27, wherein the modified cellulose is carboxymethyl cellulose, methyl cellulose, and/or hydroxypropylmethyl cellulose.

Embodiment 29: The composition of embodiment 27 or embodiment 28, wherein the soluble decellularized tissue components are derived from at least one of intervertebral disc, tendon, ligaments, cartilage, bone, cardiac tissues, and/or vascular tissues.

Embodiment 30: The composition of embodiment 29, wherein the soluble decellularized tissue components are dissolved in growth media.

Embodiment 31: The composition of any one of the foregoing embodiments, wherein the embedded phase has a viscosity between 10 and 1×10⁷ Pa-S.

Embodiment 32: The composition of any one of the foregoing embodiments, wherein the at least one cell comprises a suspending living cell, allogenic mesenchymal stem cell, and/or induced pluripotent stem cell.

Embodiment 33: The composition of embodiment 32, wherein the induced pluripotent stem cell is capable of inducing at least one cellular process related to nucleus pulposus, annulus fibrosus, hyaline cartilage, elastic cartilage, fibrocartilage, tendon, ligament, long bones, short bones, flat bone, irregular bones, cardiac tissue, or vascular tissue.

Embodiment 34: The composition of any one of the foregoing embodiments, wherein the embedded phase comprises cylindrical channels.

Embodiment 35: The composition of embodiment 34, wherein the channels are from about 10 μm to about 1000 μm in diameter.

Embodiment 36: The composition of any one of the foregoing embodiments, wherein the embedded phase comprises spherical compartments.

Embodiment 37: The composition of embodiment 36, wherein the spherical compartments are connected within the support phase.

Embodiment 38: The composition of embodiment 36, wherein the spherical compartments are not connected within the support phase.

Embodiment 39: The composition of any one of the foregoing embodiments, wherein the embedded phase is deposited on top of the support phase.

Embodiment 40: The composition of any one of embodiments 1-38, wherein the embedded phase is deposited near the top of the support phase.

Embodiment 41: A method for culturing a cell, wherein the method comprises: a. providing a thermosensitive hydrogel composition, wherein the thermosensitive hydrogel composition comprises: i. a support phase comprising: 1. a primary component, wherein the primary component comprises a continuous or granular polymeric phase dissolved in an aqueous solution, further wherein the primary component is capable of Bingham plastic rheological behavior in the presence of an aqueous media: 2. a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b. depositing an embedded phase into the support phase at or below the CST of the biocompatible thermally-desolubilizable polymer or co-polymer of the support phase, wherein the embedded phase comprises at least one uncrosslinked polymer and further comprises a cell; c. culturing the at least one cell, wherein during the culturing, the temperature of the composition is increased to at or above the CST of the biocompatible thermally-desolubilizable polymer or co-polymer of the support phase following deposition of the embedded phase; and d. optionally collecting the secretome expressed by the cell.

Embodiment 42: The method of embodiment 41, wherein the cell comprises a single cell.

Embodiment 43: The method of embodiment 41, wherein the cell comprises more than one cell.

Embodiment 44: The method of any one of embodiments 41-43, wherein the temperature of the hydrogel composition comprising the embedded phase is decreased to below the CST during the culturing phase.

Embodiment 45: The method of any one of embodiments 41-44, wherein the temperature of the hydrogel composition comprising the embedded phase is cycled between the temperature being greater than or equal to the CST and the temperature being less than the CST at least once during the culturing phase.

Embodiment 46: The method of embodiment 45, wherein the temperature is cycled between at least 1 time and at least 1,000 times during the culturing phase.

Embodiment 47: The method of embodiment 45, wherein the temperature is cycled not more than 3 times per week of the culture phase.

Embodiment 48: The method of any one of embodiments 45-47, wherein the time period for each cycle is between about 15 minutes to about 5 hours.

Embodiment 49: The method of any one of embodiments 41-48, wherein a secretome expressed by the cell is collected.

Embodiment 50: The method of any one of embodiments 41-49, wherein lowering the temperature below the CST promotes reversible de-adhesion of proteins and cells from the support phase.

Embodiment 51: The method of any one of embodiments 41-50, wherein the temperature is cycled between about 4° C. and about 37° C.

Embodiment 52: The method of embodiment 51, wherein the temperature is cycled between about 25° C. and about 37° C.

Embodiment 53: The method of any one of embodiments 41-52, wherein the support phase comprises a homogenous mixture of primary and secondary components dissolved in aqueous solution below the CST of the secondary component.

Embodiment 54: The method of any one of embodiments 41-53, wherein the continuous or granular polymeric phase of the support phase comprises a biocompatible polymeric material.

Embodiment 55: The method of any one of embodiments 41-54, wherein the biocompatible thermally-desolubilizable polymer or co-polymer is in a wetted and/or rod-like state below the CST.

Embodiment 56: The method of any one of embodiments 41-55, wherein the secondary component is released or resistant to cell and protein adhesion below the CST.

Embodiment 57: The method of any one of embodiments 41-56, wherein the biocompatible thermally-desolubilizable polymer or co-polymer is in a globular, hydrophobic, gelated form at or above the CST.

Embodiment 58: The method of any one of embodiments 41-57, wherein the secondary component is adhesive for cells and proteins at or above the CST.

Embodiment 59: The method of any one of embodiments 41-58, wherein the at least one uncrosslinked polymer of the embedded phase comprises a biocompatible polymer.

Embodiment 60: The method of any one of embodiments 41-59, wherein the at least one uncrosslinked polymer of the embedded phase is dissolved in growth media.

Embodiment 61: The method of any one of embodiments 41-60, wherein the embedded phase is distributed across the support phase in spatially segregated compartments.

Embodiment 62: The method of embodiment 61, wherein the spatially segregated compartments are spherical or are cylindrical.

Embodiment 63: The method of embodiment 61 or embodiment 62, wherein the spatially segregated compartments are continuous or discontinuous.

Embodiment 64: The method of any one of embodiments 41-63, wherein the spatially defined compartments are anisotropic.

Embodiment 65: The method of any one of embodiments 41-64, wherein the overall concentration of the continuous or granular polymeric phase dissolved in an aqueous solution of the support phase is from about 0.10% to about 90%.

Embodiment 66: The method of any one of embodiments 41-65, wherein the continuous or granular polymeric phase dissolved in an aqueous solution is selected from the group consisting of poly(ethylene oxides), poly(propylene oxides), copolymers of PEO and polylactic acid (PLA), polyvinyl alcohol, celluloses, agar, agarose, chitosan, alginate, collagen, celluloses, polyacrylic acid, hyaluronates, keratins, and decellularized tissue components from at least one of intervertebral disc, tendon, ligaments, cartilage, bone, cardiac tissues, and vascular tissues.

Embodiment 67: The method of any one of embodiments 41-66, wherein the secondary component of the support phase comprises at least one material that is distinct from the primary component of the support phase.

Embodiment 68: The method of any one of embodiments 41-67, wherein the overall concentration of the secondary component is about 1% to about 25%.

Embodiment 69: The method of any one of embodiments 41-68, wherein the secondary component comprises at least one polymer selected from the group consisting of poly(N-isopropyl acrylamides) (PNIPAAm), poly(N,N-diethylacrylamide), poly(Nvinylcaprolactam), poly(2-oxazolines), poly(2-dimethylamino)ethyl methacrylate), and/or poloxamers ((poly(ethylene oxide) (PEO)-b-poly(propylene oxide)-b-PEO).

Embodiment 70: The method of any one of embodiments 41-69, wherein the biocompatible thermally-desolubilizable polymer or co-polymer of the secondar component is covalently linked to a polymer selected from the group consisting of PEO, polylactic-coglycolic acid, alginate, hyaluronic acid, gelatin, collagen, chondroitin sulfate, and decellularized extracellular matrix components.

Embodiment 71: The method of embodiment 70, wherein the decellularized extracellular matrix components are derived from tendon, ligament, bone, cartilage, intervertebral disc, vascular tissues, and/or cardiac tissues.

Embodiment 72: The method of any one of embodiments 41-71, wherein the secondary component comprises PNIPAAm.

Embodiment 73: The method of embodiment 72, wherein the PNIPAAm is covalently linked to chondroitin sulfate.

Embodiment 74: The method of any one of embodiments 41-73, wherein the secondary component comprises PNIPAAm at an overall concentration of about 1% to about 25%.

Embodiment 75: The method of any one of embodiments 41-74, wherein the support phase comprises a yield point of 50-1000 Pa below the normal body temperature of the mammal.

Embodiment 76: The method of any one of embodiments 41-75, wherein the support phase exhibits at least 25% greater elastic modulus above the CST of the secondary component.

Embodiment 77: The method of any one of embodiments 41-76, wherein the embedded phase comprises at least one of gelatin, collagen, chitosan, alginate, collagen, cellulose, modified celluloses, and soluble decellularized tissue components.

Embodiment 78: The method of embodiment 77, wherein the modified cellulose is carboxymethyl cellulose, methyl cellulose, and/or hydroxypropylmethyl cellulose.

Embodiment 79: The method of embodiment 77 or embodiment 78, wherein the soluble decellularized tissue components are derived from at least one of intervertebral disc, tendon, ligaments, cartilage, bone, cardiac tissues, and vascular tissues.

Embodiment 80: The method of embodiment 79, wherein the soluble decellularized tissue components are dissolved in growth media.

Embodiment 81: The method of any one of embodiments 41-80, wherein the embedded phase has a viscosity between about 10 and about 1×10⁷ Pa-S.

Embodiment 82: The method of any one of embodiments 41-81, wherein the at least one cell comprises suspending living cells, allogenic mesenchymal stem cells, and/or induced pluripotent stem cells.

Embodiment 83: The method of embodiment 82, wherein the induced pluripotent stem cells are capable of inducing at least one cellular process related to nucleus pulposus, annulus fibrosus, hyaline cartilage, elastic cartilage, fibrocartilage, tendon, ligament, long bones, short bones, flat bone, irregular bones, cardiac tissue, or vascular tissue.

Embodiment 84: The method of any one of embodiments 41-83, wherein the embedded phase comprises cylindrical channels.

Embodiment 85: The method of embodiment 84, wherein the channels are are from about 10 μm to about 1000 μm in diameter.

Embodiment 86: The method of any one of embodiments 41-85, wherein the embedded phase comprises spherical compartments.

Embodiment 87: The method of embodiment 86, wherein the spherical compartments are connected within the support phase.

Embodiment 88: The method of embodiment 86, wherein the spherical compartments are not connected within the support phase.

Embodiment 89: The method of any one of embodiments 41-88, wherein the method further comprises d. collecting the secretome expressed by the at least one cell.

Embodiment 90: A method of collecting a secretome of a cell, wherein the method comprises: a. providing a thermosensitive hydrogel composition, wherein the thermosensitive hydrogel composition comprises: i. a support phase comprising: 1. a primary component, wherein the primary component comprises a continuous or granular polymeric phase dissolved in an aqueous solution, further wherein the primary component is capable of Bingham plastic rheological behavior in the presence of an aqueous media; 2. a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST: b. depositing an embedded phase into the support phase at or below the CST of the biocompatible thermally-desolubilizable polymer or co-polymer of the support phase, wherein the embedded phase comprises at least one uncrosslinked polymer and further comprises a cell; c. culturing the at least one cell, wherein during the culturing, the temperature of the composition is increased to at or above the CST of the biocompatible thermally-desolubilizable polymer or co-polymer of the support phase following deposition of the embedded phase; and d. collecting the secretome expressed by the at least one cell.

Embodiment 91: A method for culturing a cell, wherein the method comprises: a. providing a thermosensitive hydrogel composition, wherein the thermosensitive hydrogel composition comprises: i. a support phase comprising: 1. a primary component, wherein the primary component comprises a continuous or granular polymeric phase dissolved in an aqueous solution, further wherein the primary component is capable of Bingham plastic rheological behavior in the presence of an aqueous media; 2. a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b. depositing an embedded phase near the top of the support phase at or below the CST of the biocompatible thermally-desolubilizable polymer or co-polymer of the support phase, wherein the embedded phase comprises at least one uncrosslinked polymer and further comprises a cell; c. culturing the at least one cell, wherein during the culturing, the temperature of the composition is increased to at or above the CST of the biocompatible thermally-desolubilizable polymer or co-polymer of the support phase following deposition of the embedded phase; and d. optionally collecting the secretome expressed by the cell.

Embodiment 92: A method for culturing a cell, wherein the method comprises: a. providing a thermosensitive hydrogel composition, wherein the thermosensitive hydrogel composition comprises: i. a support phase comprising: 1. a primary component, wherein the primary component comprises a continuous or granular polymeric phase dissolved in an aqueous solution, further wherein the primary component is capable of Bingham plastic rheological behavior in the presence of an aqueous media; 2. a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST; b. depositing an embedded phase on top of the support phase at or below the CST of the biocompatible thermally-desolubilizable polymer or co-polymer of the support phase, wherein the embedded phase comprises at least one uncrosslinked polymer and further comprises a cell; c. culturing the at least one cell, wherein during the culturing, the temperature of the composition is increased to at or above the CST of the biocompatible thermally-desolubilizable polymer or co-polymer of the support phase following deposition of the embedded phase; and d. optionally collecting the secretome expressed by the cell.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A thermosensitive hydrogel composition comprising:
(a) a support phase, wherein the support phase comprises:
(i) a primary component comprising a continuous or granular polymeric phase dissolved in an aqueous solution, further wherein the primary component is capable of Bingham plastic rheological behavior in the presence of an aqueous media;
(ii) a secondary component comprising a biocompatible thermally-desolubilizable polymer or co-polymer that exists in an extended form below a critical solution temperature (CST) that is lower than the normal body temperature of a mammal and in a condensed form at or above the CST;
(b) an embedded phase, wherein the embedded phase comprises at least one uncrosslinked polymer and further comprises at least one cell, wherein the embedded phase is capable of extrusion into the support phase at or below the CST of the support phase.

2. The composition of claim 1, wherein:
(a) the support phase comprises a homogenous mixture of primary and secondary components dissolved in aqueous solution below the CST of the secondary component;
(b) the continuous or granular polymeric phase of the support phase comprises a biocompatible polymeric material
(c) the biocompatible thermally-desolubilizable polymer or co-polymer is in a wetted and/or rod-like state below the CST;
(d) the secondary component is capable of releasing or being resistant to cell and protein adhesion below the CST;
(e) the biocompatible thermally-desolubilizable polymer or co-polymer is in a globular, hydrophobic, gelated form at or above the CST;
(f) the secondary component is capable of being adhesive for cells and proteins at or above the CST;
(g) the at least one uncrosslinked polymer of the embedded phase comprises a biocompatible polymer; or
(h) the at least one uncrosslinked polymer of the embedded phase is dissolved in growth media.

3-9. (canceled)

10. The composition of claim 1, wherein the embedded phase is distributed across the support phase in spatially segregated compartments.

11. The composition of claim 10, wherein:
(a) the spatially segregated compartments are spherical or cylindrical;
(b) the spatially segregated compartments are continuous;
(c) the spatially segregated compartments are discontinuous; or
(d) the spatially defined compartments are anisotropic.

12-14. (canceled)

15. The composition of claim 1, wherein:
(a) the overall concentration of the continuous or granular polymeric phase dissolved in an aqueous solution of the support phase is from about 0.1% to about 90%;
(b) the continuous or granular polymeric phase dissolved in an aqueous solution is selected from the group consisting of poly(ethylene oxides), poly(propylene oxides), copolymers of PEO and polylactic acid (PLA), polyvinyl alcohol, celluloses, agar, agarose, chitosan, alginate, collagen, celluloses, polyacrylic acid, hyaluronates, keratins, and decellularized tissue components from any one of intervertebral disc, tendon, ligaments, cartilage, bone, cardiac tissues, and vascular tissues;
(c) the secondary component of the support phase comprises at least one material that is distinct from the primary component of the support phase;
(d) the overall concentration of the secondary component is about 1% to about 25%; or
(e) the secondary component comprises at least one polymer selected from the group consisting of poly(N-isopropyl acrylamides) (PNIPAAm), poly(N,N-diethylacrylamide), poly(N-vinylcaprolactam), poly(2-oxazolines), poly(2-dimethylamino)ethyl methacrylate), and poloxamers ((poly(ethylene oxide) (PEO)-b-poly(propylene oxide)-b-PEO).

16-19. (canceled)

20. The composition of claim 1, wherein the biocompatible thermally-desolubilizable polymer or co-polymer of the secondary component is covalently linked to a polymer selected from the group consisting of PEO, polylactic-coglycolic acid, alginate, hyaluronic acid, gelatin, collagen, chondroitin sulfate, and decellularized extracellular matrix components.

21. (canceled)

22. The composition of claim 1, wherein the secondary component comprises PNIPAAm.

23. The composition of claim 22, wherein the PNIPAAm is covalently linked to chondroitin sulfate.

24. The composition of claim 1, wherein:
(a) the secondary component comprises PNIPAAm at an overall concentration of about 1% to about 25%;
(b) the support phase comprises a yield point of 50-1000 Pa below the normal body temperature of the mammal; or
(c) the support phase exhibits at least 25% greater elastic modulus above the CST of the secondary component.

25-26. (canceled)

27. The composition of claim 1, wherein the embedded phase comprises at least one of gelatin, collagen, chitosan, alginate, collagen, cellulose, modified celluloses, and soluble decellularized tissue components

28. The composition of claim 27, wherein:
(a) the modified cellulose is carboxymethyl cellulose, methyl cellulose, and/or hydroxypropylmethyl cellulose; or
(b) the soluble decellularized tissue components are derived from at least one of intervertebral disc, tendon, ligaments, cartilage, bone, cardiac tissues, and/or vascular tissues.

29-30. (canceled)

31. The composition of claim 1, wherein:
(a) the embedded phase has a viscosity between 10 and 1×10⁷ Pa-S;
(b) the at least one cell comprises a suspending living cell, allogenic mesenchymal stem cell, and/or induced pluripotent stem cell;
(c) the embedded phase comprises cylindrical channels;
(d) the embedded phase is deposited on top of the support phase; or
(e) the embedded phase is deposited near the top of the support phase.

32-35. (canceled)

36. The composition of claim 1, wherein the embedded phase comprises spherical compartments.

37. The composition of claim 36, wherein:
(a) the spherical compartments are connected within the support phase; or
(b) the spherical compartments are not connected within the support phase.

38-40. (canceled)

41. A method for culturing a cell, wherein the method comprises:
(a) providing a thermosensitive hydrogel composition, wherein the thermosensitive hydrogel composition comprises:
(i) a support phase comprising:
(1) a primary component, wherein the primary component comprises a continuous or granular polymeric phase dissolved in an aqueous solution, further wherein the primary component is capable of Bingham plastic rheological behavior in the presence of an aqueous media;
(2) a secondary component, wherein the secondary component comprises a biocompatible thermally-desolubilizable polymer or co-polymer that exists in an extended form below a critical solution temperature (CST) that is lower than the normal body temperature of the mammal and in a condensed form at or above the CST;
(b) depositing an embedded phase into the support phase at or below the CST of the biocompatible thermally-desolubilizable polymer or co-polymer of the support phase, wherein the embedded phase comprises at least one uncrosslinked polymer and further comprises a cell;
(c) culturing the at least one cell, wherein during the culturing, the temperature of the composition is increased to at or above the CST of the biocompatible thermally-desolubilizable polymer or co-polymer of the support phase following deposition of the embedded phase;
(d) optionally collecting the secretome expressed by the cell.

42. The method of claim 41, wherein;
(a) the cell comprises a single cell;
(b) the cell comprises more than one cell;
(c) the temperature of the hydrogel composition comprising the embedded phase is decreased to below the CST during the culturing phase;
(d) a secretome expressed by the cell is collected;
(e) lowering the temperature below the CST promotes reversible de-adhesion of proteins and cells from the support phase;
(f) the temperature is cycled between about 4° C. and about 37° C.; or
(g) the method further comprises (d) collecting the secretome expressed by the at least one cell.

43-44. (canceled)

45. The method of claim 41, wherein the temperature of the hydrogel composition comprising the embedded phase is cycled between the temperature being greater than or equal to the CST and the temperature being less than the CST at least once during the culturing phase.

46. The method of claim 45, wherein:
(Lathe temperature is cycled between at least 1 time and at least 1,000 times during the culturing phase;
(b) the temperature is cycled not more than 3 times per week of the culture phase; or
(c) the time period for each cycle is between about 15 minutes to about 5 hours.

47-60. (canceled)

61. The method claim 41, wherein the embedded phase is distributed across the support phase in spatially segregated compartments.

62. The method of claim 61, wherein:
a) the spatially segregated compartments are spherical or are cylindrical; or
(b) the spatially segregated compartments are continuous or discontinuous.

63-92. (canceled)
